(19) United States
(12) Patent Application Publication
(10) Pub. No.: US 2024/0353769 A1
(43) Pub. Date: Oct. 24, 2024

(54) ELECTROPHOTOGRAPHIC PHOTORECEPTOR, COATING LIQUID FOR FORMING ELECTROPHOTOGRAPHIC PHOTORECEPTOR PROTECTIVE LAYER, COMPOUND, ELECTROPHOTOGRAPHIC PHOTORECEPTOR CARTRIDGE, AND IMAGE FORMATION DEVICE

(71) Applicant: MITSUBISHI CHEMICAL CORPORATION, Tokyo (JP)

(72) Inventors: Tsukasa HASEGAWA, Tokyo (JP); Manuel Emilio OTERO RAMIREZ, Tokyo (JP); Akira ANDO, Tokyo (JP); Hideki GOROUMARU, Tokyo (JP)

(73) Assignee: MITSUBISHI CHEMICAL CORPORATION, Tokyo (JP)

(21) Appl. No.: 18/754,098

(22) Filed: Jun. 25, 2024

Related U.S. Application Data
(63) Continuation of application No. PCT/JP2022/047853, filed on Dec. 26, 2022.

(30) Foreign Application Priority Data
Dec. 28, 2021 (JP) 2021-214076
Dec. 28, 2021 (JP) 2021-214077

(51) Int. Cl. G03G 5/07; G03G 5/047; G03G 15/00

(52) U.S. Cl. CPC G03G 5/071; G03G 5/047; G03G 15/75; G03G 2221/1606

(57) ABSTRACT

A compound represented by the formula (1) below is proposed as a novel compound having electron-transporting property which dissolves sufficiently in an organic solvent.

one or more substituents one or more substituents one or more substituents one or more substituents one or more substituents one or more substituents one or more substituents one or more substituents one or more substituents one or more substituents one or more substituents one or more substituents one or more substituents one or more substituents one or more substituents one or more substituents

### TECHNICAL FIELD

The present invention relates to an electrophotographic photoreceptor used for a copier, a printer or the like, a coating liquid for forming an electrophotographic photoreceptor protective layer for forming a protective layer of the electrophotographic photoreceptor and an electrophotographic photoreceptor cartridge and an image formation device using the electrophotographic photoreceptor. The present invention also relates to a compound, specifically a compound having electron-transporting property, such as a compound which is useful as an electron-transporting compound or the like that is a raw material of an electrophotographic photoreceptor used for a copier, a printer or the like.

### BACKGROUND ART

In a printer, a copier and the like, when light is applied to a charged organic photoconductor (OPC) drum, an electrostatic latent image is formed because the charge is eliminated from the part, and an image can be obtained because the toner adheres to the electrostatic latent image. Thus, in a device using electrophotographic technique, the photoreceptor is the key member.

Because there is considerable room for material selection for such a kind of organic photoconductor and because the characteristics of the photoreceptor are easily regulated, a “function-separated photoreceptor” in which the functions of generating and transporting the charge are assigned to different compounds has become the mainstream. For example, a single-layer electrophotographic photoreceptor (called a single-layer photoreceptor below) having a charge generation material (CGM) and a charge transport material (CTM) in the same layer and a laminate-type electrophotographic photoreceptor (called a laminate-type photoreceptor below) obtained by laminating a charge generation layer containing a charge generation material (CGM) and a charge transport layer containing a charge transport material (CTM) are known. Moreover, the charging methods of a photoreceptor include a negatively charging method for negatively charging the surface of a photoreceptor and a positively charging method for positively charging the surface of a photoreceptor.

Combinations of the layer configuration of a photoreceptor and the charging method which are currently used are “a negatively charged laminate-type photoreceptor” and “a positively charged single-layer photoreceptor”.

A general “negatively charged laminate-type photoreceptor” has a configuration obtained by providing an undercoat layer (UCL) composed of a resin or the like on a conductive base such as an aluminum tube, providing a charge generation layer (CGL) composed of a charge generation material (CGM), a resin and the like thereon and further providing a charge transport layer (CTL) composed of a hole transport material (HTM), a resin and the like thereon.

On the other hand, a general “positively charged single-layer photoreceptor” has a configuration obtained by providing an undercoat layer (UCL) composed of a resin or the like on a conductive base such as an aluminum tube and providing a single-layer photosensitive layer composed of a charge generation material (CGM), a hole transport material (HTM), an electron transport material (ETM), a resin and the like thereon (for example, see PTL 1).

In both photoreceptors, by charging the surface of the photoreceptor by a corona discharging method or a contact method and then exposing the photoreceptor to neutralize the charge on the surface, an electrostatic latent image is formed due to the potential difference from the surrounding surface. Then, a toner is brought into contact with the photoreceptor surface to form a toner image corresponding to the electrostatic latent image, and print is finished by transferring/heat-melt fixing the image on paper or the like.

As described above, a photosensitive layer is formed on a conductive support in the basic configuration of an electrophotographic photoreceptor, but a protective layer is sometimes provided on the photosensitive layer for the purpose of improving the abrasion resistance or the like.

As a technique for improving the mechanical strength or the abrasion resistance of the surface of a photoreceptor, a photoreceptor obtained by forming a layer containing a compound having a chain-polymerizable functional group as the outermost layer of the photoreceptor and applying energy such as heat, light and radiation to the layer to polymerize the compound to form a cured resin layer is disclosed (for example, see PTLs 1 and 2).

### CITATION LIST

### Patent Literature

PTL 1: U.S. Pat. No. 9,417,538B
PTL 2: WO2010/035683 (JP5263296B)

### SUMMARY OF INVENTION

### Technical Problem

As described above, a protective layer is provided to improve the abrasion resistance of the photoreceptor. In particular, a protective layer using a curable compound has particularly excellent mechanical strength.

Moreover, excellent electron-transporting property as well as mechanical strength is required for such a protective layer to improve the electrical property of the photoreceptor. As means therefor, it is believed to be effective to add a compound having an electron-transporting structure (also referred to as “an electron-transporting compound”) to a protective layer. Specifically, a protective layer is generally formed by dissolving a curable composition containing a compound having electron-transporting property in an organic solvent to produce a coating liquid for forming a protective layer and coating the surface of the photoreceptor with the coating liquid for forming a protective layer.

It has been found, however, that the electron-transporting compounds include those which do not dissolve in an organic solvent sufficiently when contained in a protective layer, do not achieve a protective layer (film) having a uniform composition due to aggregation or precipitation of the electron-transporting compounds and have insufficient electrical property.

Thus, an object of the present invention is to provide a novel electrophotographic photoreceptor having a photosensitive layer and a protective layer in this order on a conductive support in which a protective layer having a uniform composition can be formed without aggregation or precipitation of the electron-transporting compound and which can further achieve an excellent electrical property, in particular excellent residual potential property, even when an electron-transporting compound is contained in the protective layer. Another object of the present invention is to provide a novel compound having electron-transporting property which dissolves sufficiently in an organic solvent.

### Solution to Problem

As a result of examination, the present inventors propose the electrophotographic photoreceptor, the electrophotographic photoreceptor cartridge, the image formation device, the coating liquid for forming an electrophotographic photoreceptor protective layer and the compound below to achieve the objects.

[1] An electrophotographic photoreceptor having at least a photosensitive layer and a protective layer in this order on a conductive support in which the protective layer contains a polymer of an electron-transporting compound represented by the following formula (1).

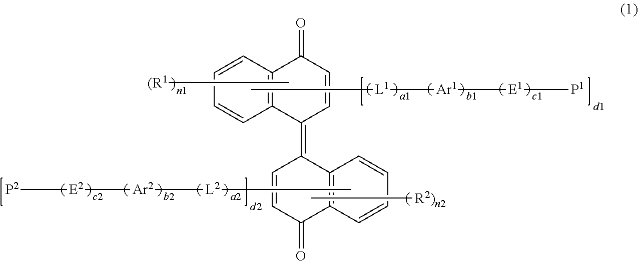

In the formula (1), L¹ and L² each independently represent a divalent group. Ar¹ and Ar² each independently represent a divalent aromatic group having 6 to 30 carbon atoms or a divalent heteroaromatic group having 3 to 30 carbon atoms. E¹ and E² each independently represent a divalent group. P¹ and P² each independently represent a chain-polymerizable functional group. R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents. a1, a2, b1, b2, c1 and c2 each independently represent an integer of 1 or more. d1 and d2 each independently represent an integer of 0 or more and 6 or less. Here, d1+d2 is one or more. n1 and n2 each independently represent an integer of 0 or more.

[2] The electrophotographic photoreceptor described in [1] above in which P¹ and P² in the formula (1) are each independently an acryloyl group or a methacryloyl group.

[3] The electrophotographic photoreceptor described in [1] or [2] above in which Ar¹ and Ar² in the formula (1) are each independently a phenylene group, a naphthylene group or a pyridylene group.

[4] The electrophotographic photoreceptor described in any one of [1] to [3] above in which L′ and L² in the formula (1) are each independently an alkylene group.

[5] The electrophotographic photoreceptor described in any one of [1] to [4] above in which E¹ and E² in the formula (1) are each independently a divalent group having an ester bond.

[6] The electrophotographic photoreceptor described in [5] above in which E¹ and E² in the formula (1) are each independently a divalent group having two or more ester bonds.

[7] The electrophotographic photoreceptor described in [6] above in which the divalent group having two or more ester bonds is the group represented by the formula (E-1) or the formula (E-2).

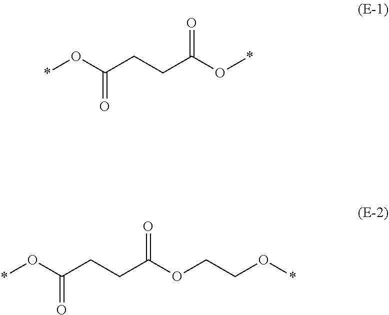 [SMILES: *OC(=O)CCC(=O)O* ; *OCCOC(=O)CCC(=O)O*]

[8] The electrophotographic photoreceptor described in any one of [1] to [7] above in which the electron-transporting compound content of the protective layer is 40 parts by mass or more based on the total mass of 100 parts by mass of the protective layer.

[9] The electrophotographic photoreceptor described in any one of [1] to [8] above which further contains a curable compound.

[10] The electrophotographic photoreceptor described in [9] above in which the ratio (mass ratio) of the curable compound to the electron-transporting compound in the protective layer is 1.0 or less.

[11] An electrophotographic photoreceptor cartridge having the electrophotographic photoreceptor described in any one of [1] to above.

[12] An image formation device having the electrophotographic photoreceptor described in any one of [1] to above.

[13] A coating liquid for forming an electrophotographic photoreceptor protective layer containing an electron-transporting compound represented by the formula (1) and a solvent.

[14] The coating liquid for forming an electrophotographic photoreceptor protective layer described in above which further contains a curable compound and in which the curable compound content is 10 parts by mass or less based on 100 parts by mass of the solvent.

[15] A compound represented by the formula (1).

[16] The compound described in above in which P¹ and P² in the formula (1) are each independently an acryloyl group or a methacryloyl group.

[17] The compound described in or above in which Ar¹ and Ar² in the formula (1) are each independently a phenylene group, a naphthylene group or a pyridylene group.

[18] The compound described in any one of to above in which L¹ and L² in the formula (1) are each independently an alkylene group.

[19] The compound described in any one of to above in which E¹ and E² in the formula (1) are each independently a divalent group having an ester bond.

[20] The compound described in above in which E¹ and E² in the formula (1) are each independently a divalent group having two or more ester bonds.

[21] The compound described in above in which the divalent group having two or more ester bonds is the group represented by the formula (E-1) or the formula (E-2).

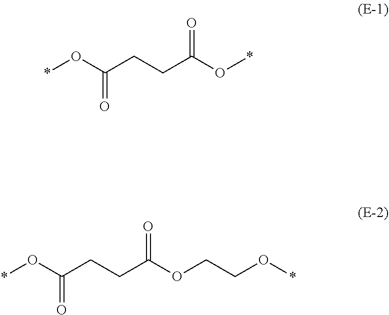 [SMILES: *OC(=O)CCC(=O)O* ; *OCCOC(=O)CCC(=O)O*]

### Advantageous Effects of Invention

In the electrophotographic photoreceptor proposed in the present invention, an electron-transporting compound having a structure in which a chain-polymerizable functional group is bonded to a dinaphthoquinone skeleton through an aromatic group, namely through an aromatic group as a spacer, is contained in the protective layer. As a result, even when a compound having an electron-transporting structure is contained in the protective layer, aggregation or precipitation of the electron-transporting compound can be suppressed, and a protective layer (film) having a uniform composition can be obtained. Furthermore, electron-transporting property can be added to the protective layer, and an excellent electrical property, in particular excellent residual potential property, can be achieved.

Moreover, the novel compound proposed in the present invention has a structure in which a chain-polymerizable functional group is bonded to a dinaphthoquinone skeleton through an aromatic group, namely through an aromatic group as a spacer, and thus has electron-transporting property and dissolves sufficiently in an organic solvent.

### BRIEF DESCRIPTION OF DRAWINGS

FIG. 1 is a FIGURE schematically illustrating an example configuration of an image formation device which can be configured using the electrophotographic photoreceptor according to an example of the present invention.

### DESCRIPTION OF EMBODIMENTS

Embodiments for carrying out the present invention (embodiments of the invention below) will be explained in detail below. Here, the present invention is not limited to the embodiments below and can be carried out with various modifications in the scope of the gist thereof.

### <<Compound of Present Invention>>

The compound according to an example of the embodiment of the present invention (referred to as “the compound of the present invention”) is preferably a compound represented by the following formula (1). In other words, the compound of the present invention is preferably a compound having a structure in which a chain-polymerizable functional group is bonded to a dinaphthoquinone skeleton through an aromatic group, namely through an aromatic group as a spacer.

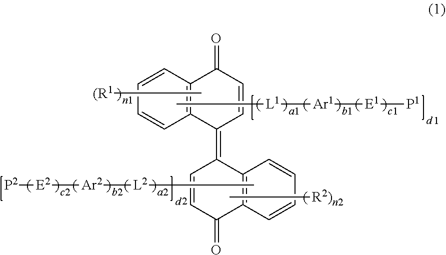

In the formula (1), L¹ and L² form a part of the spacers which bind the dinaphthoquinone skeleton and the chain-polymerizable functional groups and are each independently a divalent group. In particular, in view of the solubility in an organic solvent, an alkylene group, an ether group, an ester group and the like are preferable, and of these, an alkylene group is more preferable.

The alkylene group is a methylene group, a methylmethylene group, an ethylene group, a propylene group, an isopropylene group, a butylene group, an isobutylene group or the like and is preferably a methylmethylene group of these. Moreover, the number of the carbon atoms of the alkylene group is preferable one or more and is preferably four or less.

In the formula (1), Ar¹ and Ar² also form a part of the spacers which bind the dinaphthoquinone skeleton and the chain-polymerizable functional groups and are preferably each independently a divalent aromatic group having 6 to 30 carbon atoms or a divalent heteroaromatic group having 3 to 30 carbon atoms.

Of these, in view of the solubility and the stability, Ar¹ and Ar² are preferably each independently a phenylene group, a naphthylene group or a pyridylene group, more preferably a phenylene group.

In the formula (1), E¹ and E² also form a part of the spacers which bind the dinaphthoquinone skeleton and the chain-polymerizable functional groups and are each independently a divalent group. In particular, in view of the solubility and the stability, an alkylene group, a divalent group having a ketone group, a divalent group having an ether bond, a divalent group having an ester bond, a divalent group in which these groups are linked and the like are preferable, and a divalent group having an ester bond is more preferable. Of divalent groups having an ester bond, a divalent group having two or more ester bonds is preferable.

The divalent group having an ester bond is preferably the group represented by the formula (E-1) or the formula (E-2) below.

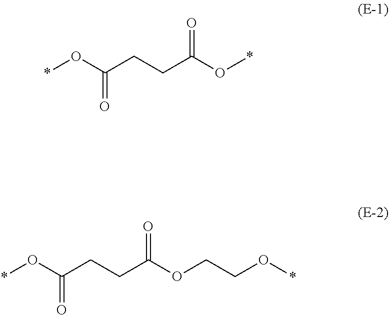 [SMILES: *OC(=O)CCC(=O)O* ; *OCCOC(=O)CCC(=O)O*]

In the formula (E-1) and the formula (E-2), * represents the binding site to Ar¹, Ar², P¹ or P².

In the formula (1), P¹ and P² are each independently a chain-polymerizable functional group.

The chain-polymerizable functional group may be a known chain-polymerizable functional group, and examples thereof include an acryloyl group, a methacryloyl group, an acrylamide group, a methacrylamide group, a styrene group and the like. Moreover, P¹ and P² may be the groups represented by the formulae (P-1) to (P-5) below.

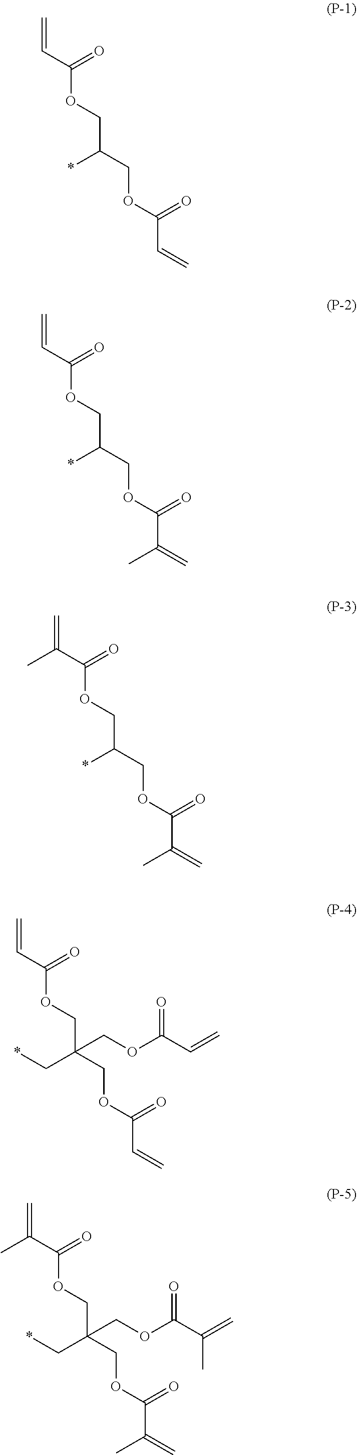

In the formula (P-1) to the formula (P-5), * represents a binding site to E¹ or E².

Of these, in view of the solubility and the stability, P¹ and P² are preferably each independently an acryloyl group, a methacryloyl group or the formula (P-3), more preferably an acryloyl group or a methacryloyl group.

The chain-polymerizable functional groups of P¹ and P² may be monofunctional or multifunctional, namely bi- or higher-functional. Of these, the chain-polymerizable functional groups are preferably monofunctional.

In the formula (1), R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents. Of these, an alkyl group which may have one or more substituents, an alkoxy group which may have one or more substituents, a haloalkyl group which may have one or more substituents, a halogen atom, a cyano group, an aromatic hydrocarbon group which may have one or more substituents and an aromatic heterocyclic group which may have one or more substituents are preferable, and of these, in view of the solubility in an organic solvent, an alkyl group which may have one or more substituents is more preferable.

Here, “which may have one or more substituents” in the present invention means that the group can have one or more substituents and has the meanings including both having one or more substituents and having no substituent.

In the compound of the present invention, the substituents of the alkyl group which may have one or more substituents and the like include an alkyl group, an alkoxy group, an aryloxy group, a heteroaryloxy group, an alkoxycarbonyl group, a dialkylamino group, a diarylamino group, an arylalkylamino group, an acyl group, a haloalkyl group, an alkylthio group, an arylthio group, a silyl group, a siloxy group, an aromatic hydrocarbon group, an aromatic heterocyclic group and the like. In view of the solubility, the substituents are preferably alkyl groups when the groups have substituents, and the groups more preferably have no substituent.

In the formula (1), a1, a2, b1, b2, c1 and c2 are each independently an integer of 1 or more, of these, in view of the solubility and the electron-transporting property, preferably 1 or more and 10 or less, of these, further preferably 1 or more or 7 or less, of these, further preferably 1 or more or 5 or less.

Of these, in view of the solubility and the electron transporting property, b1, which indicates the repeating number of Ar¹, and b2, which indicates the repeating number of Ar², are preferably 1 or more and 3 or less, of these, further preferably 1 or more or 2 or less, of these, further preferably 1.

In the formula (1), d1 and d2 are each independently an integer of 0 or more and 6 or less, of these, in view of the solubility and the stability, preferably 1 or more and 4 or less, of these, further preferably 1 or more and 3 or less, of these, further preferably 1 or more and 2 or less, of these, further preferably 1. Here, d1+d2 is one or more.

In the formula (1), n1 and n2 are each independently an integer of 0 or more, of these, in terms of the solubility and the stability, preferably 6 or less, of these, further preferably 3 or less, of these, further preferably 1 or less. Moreover, the compound of the present invention may contain an optical isomer but is more preferably a trans isomer.

### Specific Examples of Compound of Present Invention

Specific examples of the compound of the present invention are shown below, but the compound is not limited to the examples.

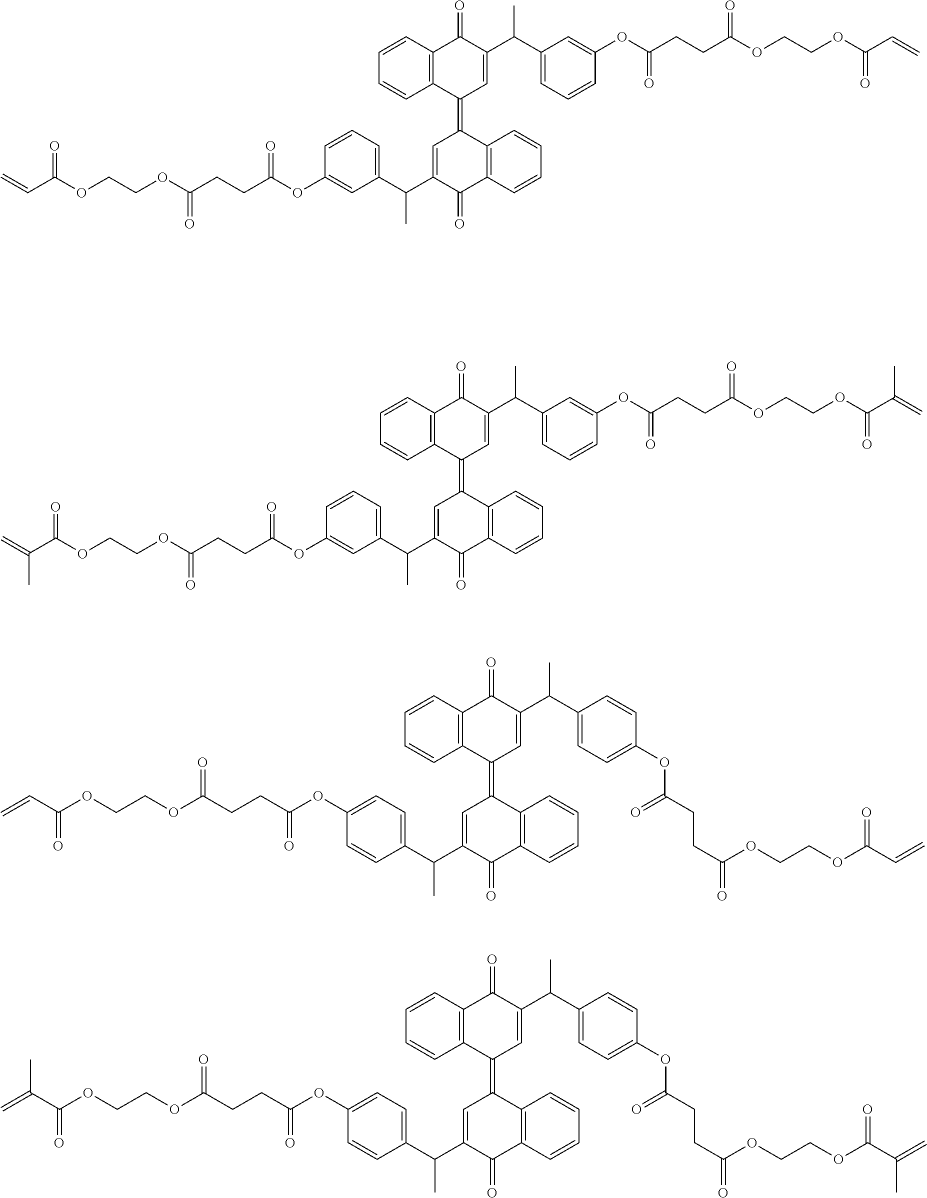

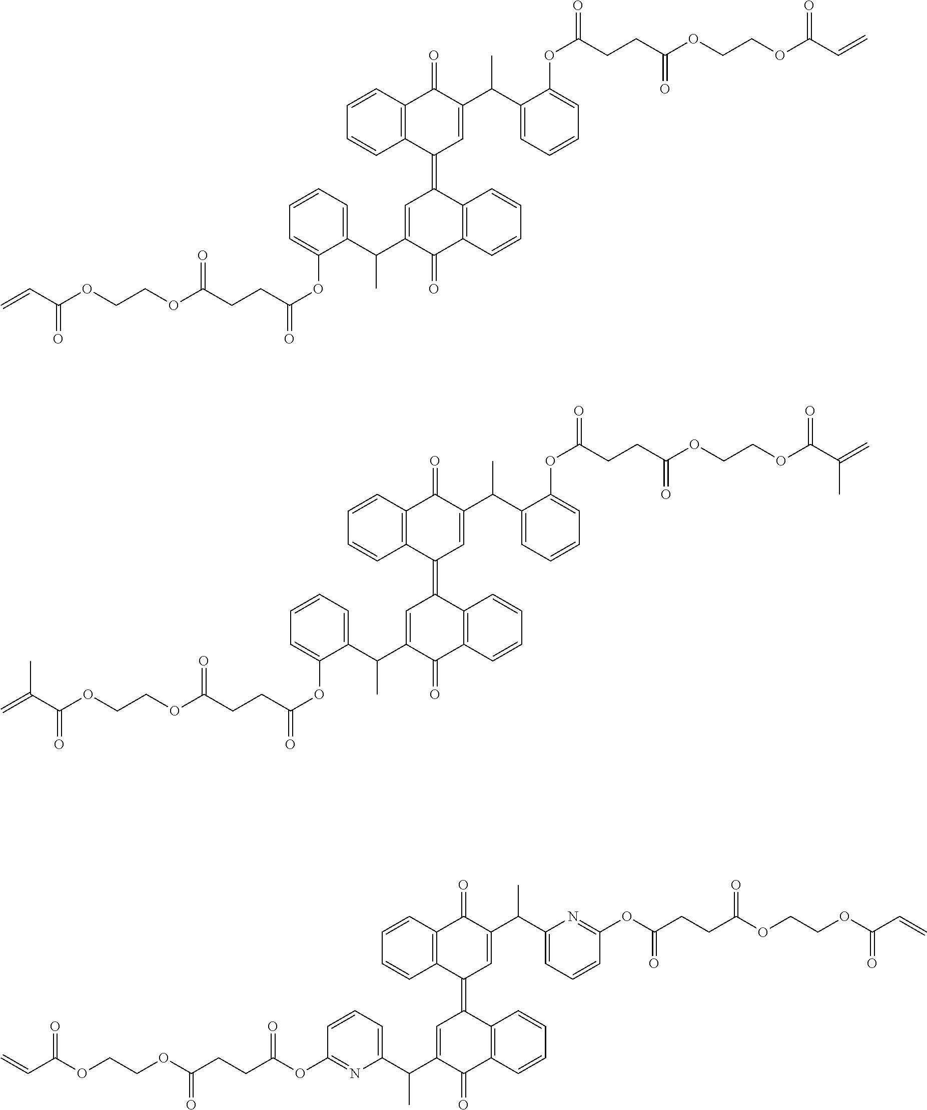

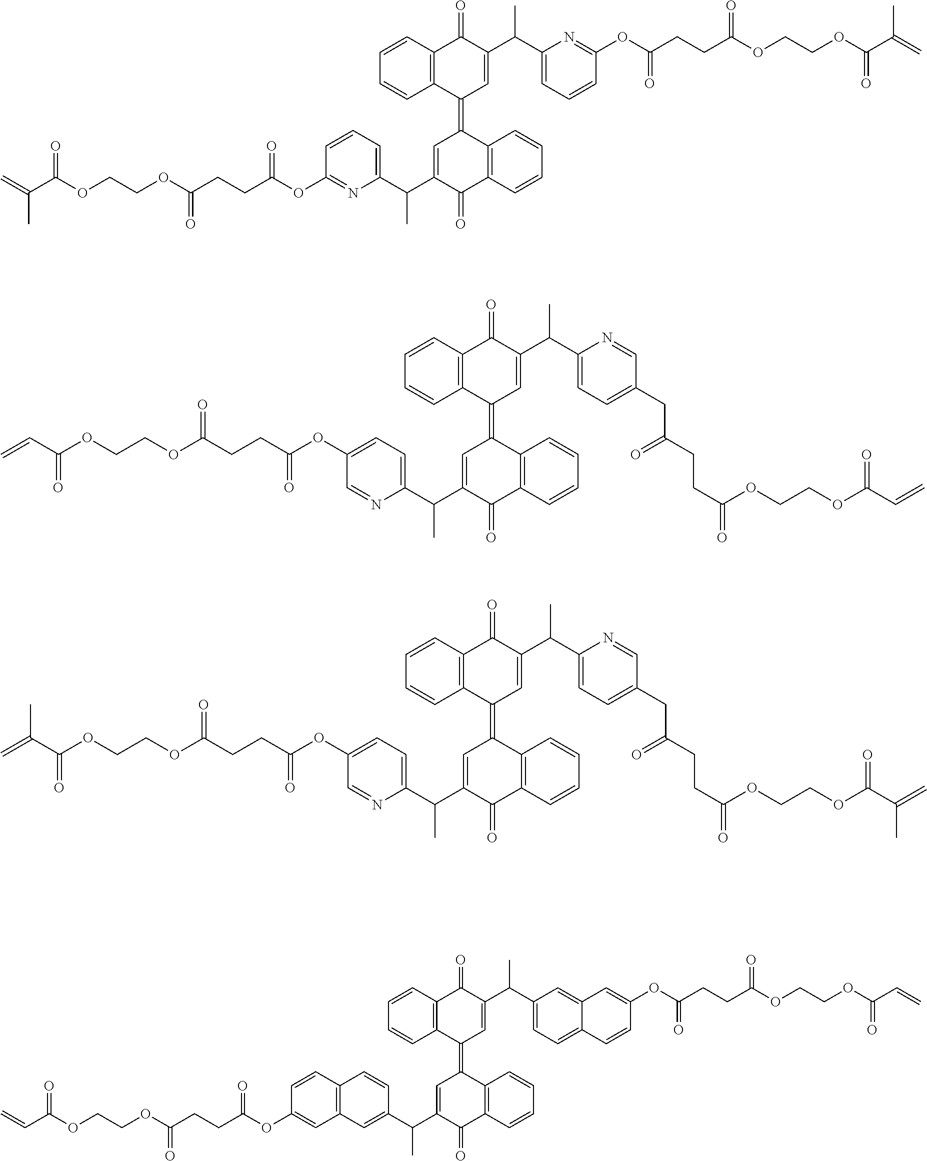

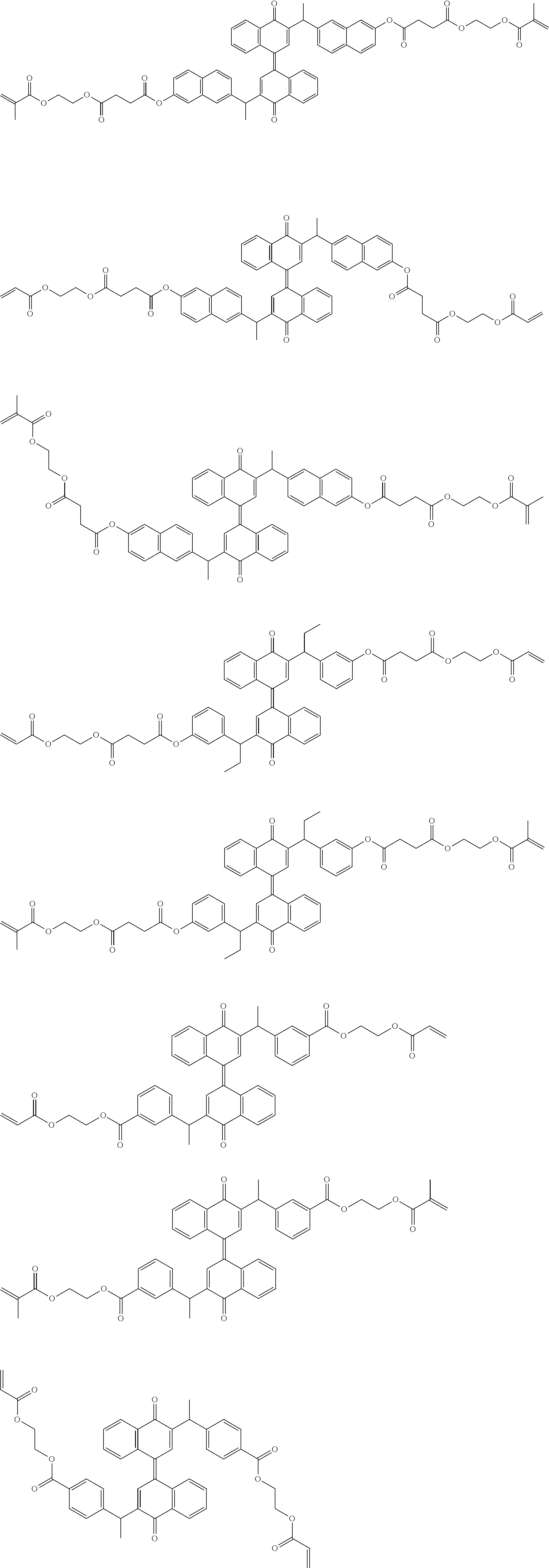

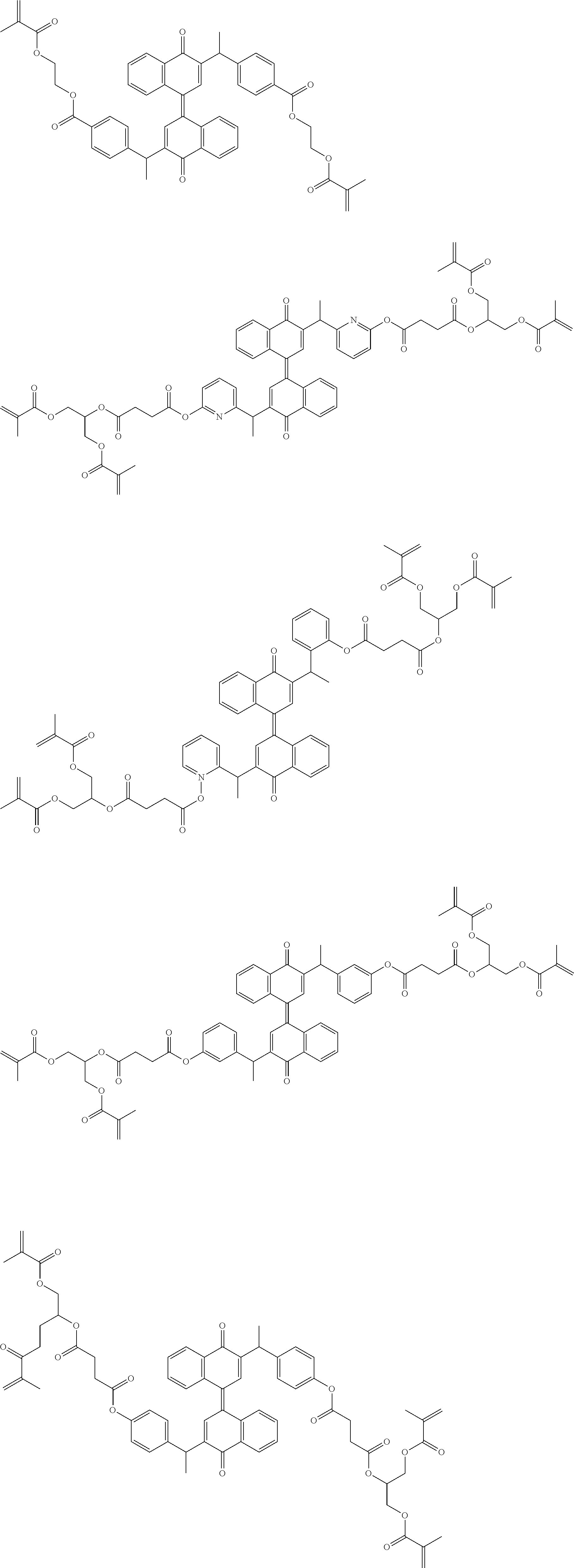

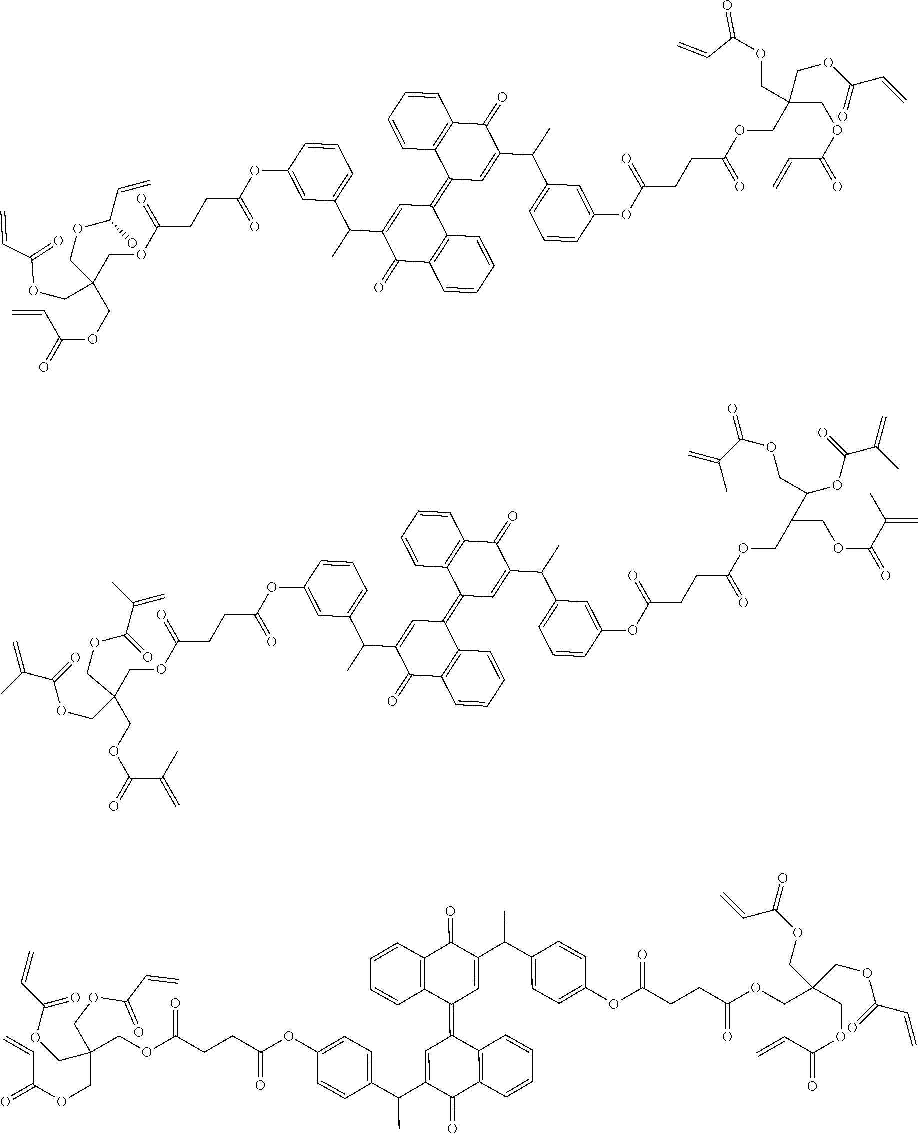

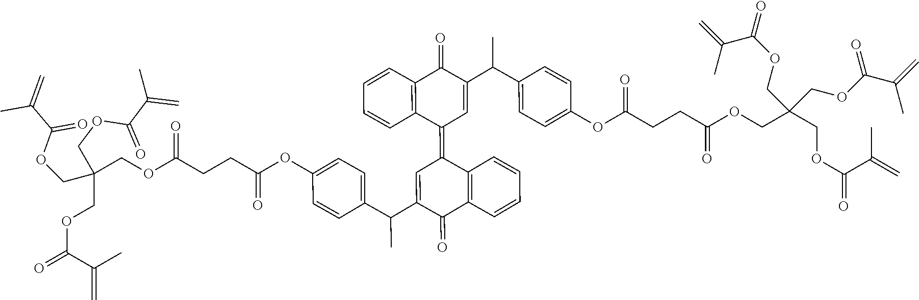 [SMILES: C=C(C)C(=O)OCC(COC(=O)CCC(=O)Oc1ccc(C(C)C2=C/C(=C3/C=C(C(C)c4ccc(OC(=O)CCC(=O)OCC(COC(=O)C(=C)C)(COC(=O)C(=C)C)COC(=O)C(=C)C)cc4)C(=O)c4ccccc43)c3ccccc3C2=O)cc1)(COC(=O)C(=C)C)COC(=O)C(=C)C]

### <<Electrophotographic Photoreceptor of Present Invention>>

The electrophotographic photoreceptor according to an example of the embodiment of the present invention (also referred to as “the electrophotographic photoreceptor of the present invention”) is an electrophotographic photoreceptor having a photosensitive layer and a protective layer in this order on a conductive support.

The electrophotographic photoreceptor of the present invention can optionally have a layer other than the photosensitive layer and the protective layer.

Moreover, the charging method of the electrophotographic photoreceptor of the present invention may be either a negatively charging method for negatively charging the surface of the photoreceptor or a positively charging method for positively charging the surface of the photoreceptor. Of these, considering that electron-transporting property is required for the protective layer, the positively charging method is preferable because the effect of the present invention is believed to be exhibited more by the positively charging method.

In the electrophotographic photoreceptor of the present invention, the side opposite to the conductive support is the upper side or the surface side, and the conductive support side is the lower side or the back surface side.

### <Protective Layer of Present Invention>

The protective layer of the present invention is preferably a layer containing a polymer of the electron-transporting compound according to an example of the embodiment of the present invention (referred to as “the electron-transporting compound of the present invention”). That is, the protective layer is preferably a layer containing a cured material obtained by curing the electron-transporting compound of the present invention. Alternatively, the protective layer is preferably a layer containing a polymer of the electron-transporting compound of the present invention and a curable compound, namely a layer containing a cured material obtained by curing the electron-transporting compound of the present invention and a curable compound.

Here, the “electron-transporting compound” means a compound having electron-transporting property, in other words, a compound having a structure having electron-transporting property, namely an electron-transporting skeleton.

The protective layer of the present invention can be formed, for example, from a composition (referred to as “the composition for forming the protective layer of the present invention”) containing the electron-transporting compound of the present invention, containing a polymerization initiator according to the need and a curable compound according to the need and further containing inorganic particles and another material according to the need. However, the protective layer of the present invention is not limited to the layer formed from such a composition.

In the electrophotographic photoreceptor proposed in the present invention, the protective layer contains the electron-transporting compound of the present invention described below, and thus, even when a compound having an electron-transporting structure is contained in the protective layer, electron-transporting property can be added to the protective layer, and an excellent electrical property, in particular excellent residual potential property, can be achieved. As a factor which can achieve an excellent electrical property in this manner, it is believed that excellent electron-transporting property is exhibited because the dinaphthoquinone skeleton that the electron-transporting compound of the present invention has has π-electron conjugated system and is a skeleton with planarity and because the electron affinity is thus high.

In the electrophotographic photoreceptor proposed in the present invention, aggregation or precipitation of the electron-transporting compound can be suppressed, and a protective layer (film) having a uniform composition can be obtained. As a factor enabling formation of such a protective layer having a uniform composition, it is believed that a uniform protective layer can be formed because the amorphousness of the electron transporting compound improves and because the solubility of the electron transporting compound in the coating liquid for forming the protective layer improves when the dinaphthoquinone skeleton which the electron-transporting compound of the present invention has is bonded to a chain-polymerizable functional group through an aromatic group, namely through an aromatic group as a spacer. Compared to an electron-transporting compound having a structure in which a dinaphthoquinone skeleton is bonded to a chain-polymerizable functional group through an alkyl group as a spacer, when an aromatic group is a spacer, the steric hindrance is caused more, resulting in suppression of aggregation of the molecules, further improvement of the amorphousness of the electron-transporting compound and further improvement of the solubility of the electron-transporting compound in the coating liquid for forming the protective layer.

### (Electron-Transporting Compound of Present Invention)

The electron-transporting compound of the present invention used for the protective layer of the present invention is preferably the compound of the present invention described above, namely the compound represented by the formula (1). In other words, the electron-transporting compound of the present invention is preferably a compound having a structure in which a chain-polymerizable functional group is bonded to a dinaphthoquinone skeleton through an aromatic group, namely through an aromatic group as a spacer.

In view of the electron-transporting property, the electron-transporting compound content of the protective layer of the present invention, based on the total mass of 100 parts by mass of the protective layer of the present invention, is preferably 40 parts by mass or more, more preferably 60 parts by mass or more, further preferably 80 parts by mass or more.

### (Curable Compound)

The electron-transporting compound of the present invention has a chain-polymerizable functional group as described above and thus can be polymerized and cured even when the composition for forming the protective layer of the present invention does not contain any curable compound. When a curable compound is contained, however, a further effect can be obtained.

The curable compound is a compound having a chain-polymerizable functional group. In particular, a monomer, an oligomer or a polymer having a radically polymerizable functional group is preferable. Of these, a curable compound having cross-linking property, in particular, a photocurable compound, is preferable. An example thereof is a curable compound having two or more radically polymerizable functional groups. A compound having one radically polymerizable functional group can also be used in combination.

The radically polymerizable functional group can be one of an acryloyl group (including an acryloyloxy group) and a methacryloyl group (including a methacryloyloxy group) or both groups.

Examples of the compounds which are preferable as the curable compound having a radically polymerizable functional group are shown below.

Examples of the monomer having an acryloyl group or a methacryloyl group include trimethylolpropane triacrylate (TMPTA), trimethylolpropane trimethacrylate, pentaerythritol triacrylate, pentaerythritol tetraacrylate, glycerol triacrylate, tris(acryloxyethyl)isocyanurate, dipentaerythritol hexaacrylate, dimethylolpropane tetraacrylate, pentaerythritol ethoxytetraacrylate, EO-modified phosphoric acid triacrylate, 2,2,5,5-tetrahydroxymethylcyclopentanone tetraacrylate, 2-hydroxy-3-acryloyloxy propylmethacrylate, polyethylene glycol diacrylate, polypropylene glycol diacrylate, polytetramethyleneglycol diacrylate, EO-modified bisphenol A diacrylate, PO-modified bisphenol A diacrylate, 9,9-bis [4-(2-acryloyloxyethoxy)phenyl]fluorene, tricyclodecane dimethanol diacrylate, decanediol diacrylate, hexanediol diacrylate, ethylene glycol dimethacrylate, polyethylene glycol dimethacrylate, EO-modified bisphenol A dimethacrylate, PO-modified bisphenol A dimethacrylate, tricyclodecane dimethanol dimethacrylate, decanediol dimethacrylate, hexanediol dimethacrylate and the like.

Moreover, examples of the oligomer and the polymer having an acryloyl group or a methacryloyl group include urethane acrylate, ester acrylate, acrylic acrylate, epoxy acrylate and the like. Of these, urethane acrylate and ester acrylate are preferable, and of these, ester acrylate is more preferable.

A kind of the compounds above can be used alone, or two or more kinds thereof can be used in combination.

In view of the electron-transporting property, the ratio (mass ratio) of the curable compound to the electron transporting compound in the protective layer of the present invention is preferably 1.0 or less, more preferably 0.5 or less, further preferably 0.1 or less.

### (Polymerization Initiator)

The polymerization initiator can be a thermal polymerization initiator, a photopolymerization initiator or the like.

Examples of the thermal polymerization initiator include peroxide-based compounds such as 2,5-dimethylhexane-2,5-dihydroperoxide and azo-based compounds such as 2,2′-azobis(isobutyronitrile).

Photopolymerization initiators can be classified into a direct cleavage type and a hydrogen extraction type based on the difference in the radical generation mechanism.

A photopolymerization initiator of the direct cleavage type generates a radical because a part of the covalent bond in the molecule is cleaved when light energy is absorbed. On the other hand, a photopolymerization initiator of the hydrogen extraction type generates a radical because the molecule in the excited state after absorbing light energy extracts hydrogen from a hydrogen donor.

Examples of the photopolymerization initiator of the direct cleavage type include acetophenone-based or ketal-based compounds such as acetophenone, 2-benzoyl-2-propanol, 1-benzoylcyclohexanol, 2,2-diethoxyacetophenone, benzyldimethylketal and 2-methyl-4′-(methylthio)-2-morpholino propiophenone, benzoin ether-based compounds such as benzoin, benzoin methylether, benzoin ethylether, benzoin isobutylether, benzoin isopropylether and O-tosylbenzoin and acylphosphine oxide-based compounds such as diphenyl(2,4,6-trimethylbenzoyl) phosphine oxide, phenylbis(2,4,6-trimethylbenzoyl) phosphine oxide and lithium phenyl(2,4,6-trimethylbenzoyl)phosphonate.

Examples of the photopolymerization initiator of the hydrogen extraction type include benzophenone-based compounds such as benzophenone, 4-benzoylbenzoic acid, 2-benzoylbenzoic acid, methyl 2-benzoylbenzoate, methyl benzoylformate, benzyl, p-anisyl, 2-benzoylnaphthalene, 4,4′-bis(dimethylamino)benzophenone, 4,4′-dichlorobenzophenone and 1,4-dibenzoylbenzene, anthraquinone-based or thioxanthone-based compounds such as 2-ethylanthraquinone, 2-isopropylthioxanthone, 2-chlorothioxanthone, 2,4-dimethylthioxanthone, 2,4-diethylthioxanthone and 2,4-dichlorothioxanthone and the like.

Examples of the other photopolymerization initiators include camphor quinone, 1-phenyl-1,2-propanedione-2-(o-ethoxycarbonyl) oxime, acridine-based compounds, triazine-based compounds, imidazole-based compounds and the like.

To efficiently absorb light energy and generate a radical, the photopolymerization initiator preferably has an absorption wavelength in the wavelength range of the light source used for the light application. In particular, an acylphosphine oxide-based compound having an absorption wavelength at a relatively long wavelength side is preferably contained.

To compensate for the curability of the surface of the protective layer of the present invention, an acylphosphine oxide-based compound and an initiator of the hydrogen extraction type are further preferably used in combination. Here, the ratio of the initiator of the hydrogen extraction type to the acylphosphine oxide-based compound is not particularly limited. The ratio to one part by mass of the acylphosphine oxide-based compound is preferably 0.1 parts by mass or more to compensate for the surface curability, and the ratio is preferably five parts by mass or less to maintain the internal curability.

Moreover, one having the effect of promoting photopolymerization can be used alone or in combination with the photopolymerization initiator. Examples of the one having the effect of promoting photopolymerization include triethanolamine, methyldiethanolamine, ethyl 4-dimethylaminobenzoate, isoamyl 4-dimethylaminobenzoate, (2-dimethylamino)ethyl benzoate, 4,4′-dimethylaminobenzophenone and the like.

One kind of polymerization initiator or a mixture of two or more kinds thereof may be used. The polymerization initiator content, based on 100 parts by mass of the total material having radically polymerizable property, is preferably 0.5 to 40 parts by mass, in particular, further preferably one part by mass or more or 20 parts by mass or less.

### (Inorganic Particles)

The protective layer of the present invention may contain inorganic particles to improve the strong exposure property or the mechanical strength or to add electron-transporting capability. However, inorganic particles do not have to be contained.

In the present invention, that inorganic particles do not have to be contained because the protective layer contains a specific electron-transporting compound is also one of the characteristics of the present invention.

Examples of the inorganic particles include a metal oxide, a metal fluoride, potassium titanate, boron nitride and the like, and any inorganic particles which can be generally used for an electrophotographic photoreceptor can be used.

As the inorganic particles, particles of one kind may be used alone, or particles of two or more kinds may also be mixed and used.

### (Other Materials)

The protective layer of the present invention may contain another material according to the need. Examples of the other material include a stabilizer (a thermal stabilizer, an ultraviolet absorber, a light stabilizer, an antioxidant or the like), a dispersant, an antistatic agent, a colorant, a lubricant and the like. One kind thereof alone or two or more kinds thereof at any ratio in any combination can be appropriately used.

### (Formation Method of Protective Layer of Present Invention)

### [Coating Liquid for Forming Protective Layer]

The protective layer of the present invention can be formed, for example, by applying a coating liquid (referred to as “the coating liquid for forming the protective layer of the present invention”) which is obtained by dissolving or dispersing a curable composition containing the electron-transporting compound of the present invention, a polymerization initiator according to the need and a curable compound according to the need and further containing inorganic particles and another material according to the need in a solvent or in a dispersant, on the photosensitive layer of the present invention and curing the coating liquid. However, the method is not limited to the method.

The coating liquid for forming the protective layer of the present invention containing the electron-transporting compound of the present invention does not have to contain the curable compound. Even when no curable compound is contained or when the curable compound content is low, because the electron-transporting compound of the present invention is used, the mechanical strength of the protective layer is sufficiently obtained, and the deterioration in the residual potential due to the addition of the curable compound can be suppressed. This, however, does not exclude the combination use of the electron-transporting compound having a chain-polymerizable functional group and the curable compound.

The electron-transporting compound used for the coating liquid for forming the protective layer of the present invention is preferably the compound represented by the formula (1).

Preferable aspects of the curable compound, the polymerization initiator, the inorganic particles and the other material used for the coating liquid for forming the protective layer of the present invention are the same as those of the respective materials used for the protective layer of the present invention.

The ratio of the curable compound to the electron-transporting compound (curable compound/electron transporting compound) in the coating liquid for forming the protective layer of the present invention is the same as the ratio of the curable compound to the electron-transporting compound d (curable compound/electron-transporting compound) in the protective layer of the present invention described above.

In view of the membrane uniformity of the protective layer, the electron-transporting compound content of the coating liquid for forming the protective layer of the present invention, based on 100 parts by mass of the solvent, is preferably four parts by mass or more, more preferably six parts by mass or more, further preferably eight parts by mass or more. On the other hand, in view of the solubility, the content based on 100 parts by mass of the solvent is preferably 14 parts by mass or less, more preferably 12 parts by mass or less, further preferably 10 parts by mass or less.

In view of the membrane uniformity of the protective layer, the curable compound content of the coating liquid for forming the protective layer of the present invention, based on 100 parts by mass of the solvent, is preferably one part by mass or more, more preferably two parts by mass or more, further preferably four parts by mass or more. On the other hand, in view of the solubility, the content based on 100 parts by mass of the solvent is preferably 10 parts by mass or less, more preferably five parts by mass or less, further preferably 0 part by mass.

As the solvent used for the coating liquid for forming the protective layer of the present invention, for example, an organic solvent can be used. Examples of the organic solvent include: alcohols such as methanol, ethanol, propanol and 2-methoxyethanol; ethers such as tetrahydrofuran, 1,4-dioxane and dimethoxyethane; esters such as methyl formate and ethyl acetate; ketones such as acetone, methylethylketone and cyclohexanone; aromatic hydrocarbons such as benzene, toluene, xylene and anisole; chlorinated hydrocarbons such as dichloromethane, chloroform, 1,2-dichloroethane, 1,1,2-trichloroethane, 1,1,1-trichloroethane, tetrachloroethane, 1,2-dichloropropane and trichloroethylene; nitrogen-containing compounds such as n-butylamine, isopropanolamine, diethylamine, triethanolamine, ethylene diamine and triethylene diamine; aprotic polar solvents such as acetonitrile, N-methylpyrrolidone, N,N-dimethylformamide and dimethylsulfoxide; and the like. A mixed solvent thereof in any combination at any ratio can also be used. Of these, in view of the solubility and the coatability, the solvent is preferably an alcohol, an ether, an aromatic hydrocarbon or an aprotic polar solvent, more preferably an alcohol, an ether or an aromatic hydrocarbon, further preferably an alcohol or an ether.

Moreover, even the organic solvent does not dissolve the materials for the protective layer of the electrophotographic photoreceptor of the present invention in a single organic solvent, the organic solvent can be used if the materials can be dissolved by making them a mixed solvent. In general, coating unevenness can be reduced when a mixed solvent is used. When a dip coating method is used in the coating method described below, a solvent in which the under layer does not dissolve is preferably selected. From this point, an alcohol is particularly preferably contained.

The ratio of the organic solvent used for the coating liquid for forming the protective layer of the present invention and the solid content depends on the coating method of the coating liquid for forming the protective layer and can be appropriately changed and used in such a manner that a more uniform coating film is formed by the coating method applied.

### [Coating Method]

The method for coating the coating liquid for forming the protective layer of the present invention is not particularly limited, and examples thereof include a spray coating method, a spiral coating method, a ring coating method, a dip coating method and the like.

After forming a coating film by the coating method, the coating film is dried. At this point, when required sufficient drying can be achieved, the temperature and the period of drying are not limited. When the protective layer is applied after air drying alone after applying the photosensitive layer, however, sufficient drying is preferably conducted by the method described in the method for forming the photosensitive layer described below.

### [Curing Method of Protective Layer of Present Invention]

The protective layer of the present invention can be formed by applying the coating liquid for forming the protective layer of the present invention and then curing by applying energy from outside. The external energy used here may be heat, light or radiation.

The method for applying heat energy may be a heating method using air, gas such as nitrogen, steam, various heating media, infrared ray or electromagnetic waves. Moreover, the heating can be conducted from the coated surface side or from the support side. The heating temperature is preferably 100° C. or higher and 170° C. or lower.

As the light energy, a UV light source such as a high-pressure mercury lamp, a metal halide lamp, an electrodeless lamp bulb and a light-emitting diode, which have emission wavelengths mainly of ultraviolet light (UV), can be used. Moreover, a visible light source can also be selected corresponding to the absorption wavelength of the chain-polymerizable compound or the photopolymerization initiator.

In view of the curability, the light irradiation amount is preferably 10 J/cm² or more, further preferably 30 J/cm² or more, particularly preferably 100 J/cm² or more. In view of the electrical property, the light irradiation amount is preferably 500 J/cm² or less, further preferably 300 J/cm² or less, particularly preferably 200 J/cm² or less.

The radiation energy may be one using electron beam (EB).

Of the energy types, in view of the easiness of the regulation of the reaction speed, the simpleness of the device and the length of the pot life, light energy is preferably used.

After curing the protective layer, to reduce the residual stress, reduce the residual radical or improve the electrical property, a heating step may be added. The heating temperature is preferably 60° C. or higher, more preferably 100° C. or higher, and is preferably 200° C. or lower, more preferably 150° C. or lower.

### (Layer Thickness)

In view of the abrasion resistance, the thickness of the protective layer of the present invention is preferably 0.5 μm or more, in particular, further preferably 1 μm or more. On the other hand, in view of the electrical property, the thickness is preferably 5 μm or less, in particular, further preferably 3 μm or less.

In view of the same points, the thickness of the protective layer of the present invention is preferably 1/50 or more of the thickness of the photosensitive layer of the present invention, in particular, more preferably 1/40 or more, in particular, further preferably 1/30 or more. On the other hand, the thickness is preferably ⅕ or less, in particular, more preferably 1/10 or less, in particular, further preferably 1/20 or less.

### <Photosensitive Layer of Present Invention>

The photosensitive layer (also referred to as “the photosensitive layer of the present invention”) in the electrophotographic photoreceptor of the present invention is a layer containing at least a charge generation material (CGM) and a charge transport material.

The photosensitive layer of the present invention may be a single-layer photosensitive layer containing both a charge generation material and a charge transport material in the same layer or may be a laminate-type photosensitive layer which is separated into a charge generation layer and a charge transport layer.

Both for the single-layer photosensitive layer and the laminate-type photosensitive layer, the mechanism of action for obtaining the effects of the present invention, namely the effects of making the electrical property excellent and obtaining a uniform protective layer (film) by adding the electron-transporting compound of the present invention to the protective layer, is the same, and thus the effects of the present invention can be exhibited both with the single-layer photosensitive layer and the laminate-type photosensitive layer.

### <Single-Layer Photosensitive Layer>

When the photosensitive layer of the present invention is a single-layer photosensitive layer, at least a charge generation material (CGM), a hole transport material (HTM) and an electron transport material (ETM) and a binder resin are preferably contained in the same layer.

### (Charge Generation Material)

As the charge generation material used for the photosensitive layer of the present invention, for example, various photoconductive materials such as inorganic photoconductive materials and organic pigments can be used. Of these, in particular, an organic pigment is preferable, and a phthalocyanine pigment and an azo pigment are more preferable.

In particular, when a phthalocyanine pigment is used as the charge generation material, specifically, a non-metal phthalocyanine, a phthalocyanine in which a metal such as copper, indium, gallium, tin, titanium, zinc, vanadium, silicon and germanium, an oxide thereof, a halogenated material thereof or the like is coordinated or the like is preferably used. Of these, X-form or τ-form non-metal phthalocyanine, A-form, B-form, D-form or another form titanyl phthalocyanine, vanadyl phthalocyanine, chloroindium phthalocyanine, chlorogallium phthalocyanine, hydroxygallium phthalocyanine and the like, which have high sensitivity, are particularly suitable.

Moreover, when an azo pigment is used, various known bisazo pigments and trisazo pigments are suitably used.

One kind of charge generation material may be used alone, or two or more kinds thereof in any combination at any ratio may be used in combination. When two or more kinds of charge generation material are used in combination, as the method for mixing the charge generation materials used in combination, the charge generation materials may be mixed later and used or mixed in the production/processing steps of the charge generation materials such as synthesis, pigment formation and crystallization and used.

In view of the electrical property, the particle size of the charge generation material is desirably small. Specifically, the particle size of the charge generation material is preferably 1 μm or less, more preferably 0.5 μm or less. The lower limit is 0.01 μm or more. Here, the particle size of the charge generation material means the particle size in the state of being contained in the photosensitive layer.

Furthermore, in view of the sensitivity, the amount of the charge generation material in the single-layer photosensitive layer is preferably 0.1 mass % or more, more preferably 0.5 mass % or more. Moreover, in view of the sensitivity and the electrostatic property, the amount is preferably 50 mass % or less, more preferably 20 mass % or less.

### (Charge Transport Material)

Charge transport materials are classified into hole transport materials mainly having hole transport capability and electron transport materials mainly having electron transport capability. However, when the photosensitive layer of the present invention is a single-layer photosensitive layer, at least a hole transport material and an electron transport material are preferably contained in the same layer.

### [Hole Transport Material]

The hole transport material (HTM) can be selected from known materials and used. Examples thereof include electron-donating materials, such as heterocyclic compounds including a carbazole derivative, an indole derivative, an imidazole derivative, an oxazole derivative, a pyrazole derivative, a thiadiazole derivative, a benzofuran derivative and the like, an aniline derivative, a hydrazone derivative, an arylamine derivative, a stilbene derivative, a butadiene derivative, an enamine derivative, a material in which two or more kinds of the compounds are bound and a polymer having a group derived from such a compound in the main chain or a side chain, and the like.

Of these, a carbazole derivative, an arylamine derivative, a stilbene derivative, a butadiene derivative, an enamine derivative and a material in which two or more kinds of the compounds are bound are preferable, and an arylamine derivative and an enamine derivative are more preferable.

One kind of hole transport material may be used alone, or two or more kinds thereof may be used at any ratio in any combination.

The structures of preferable hole transport materials are exemplified below.

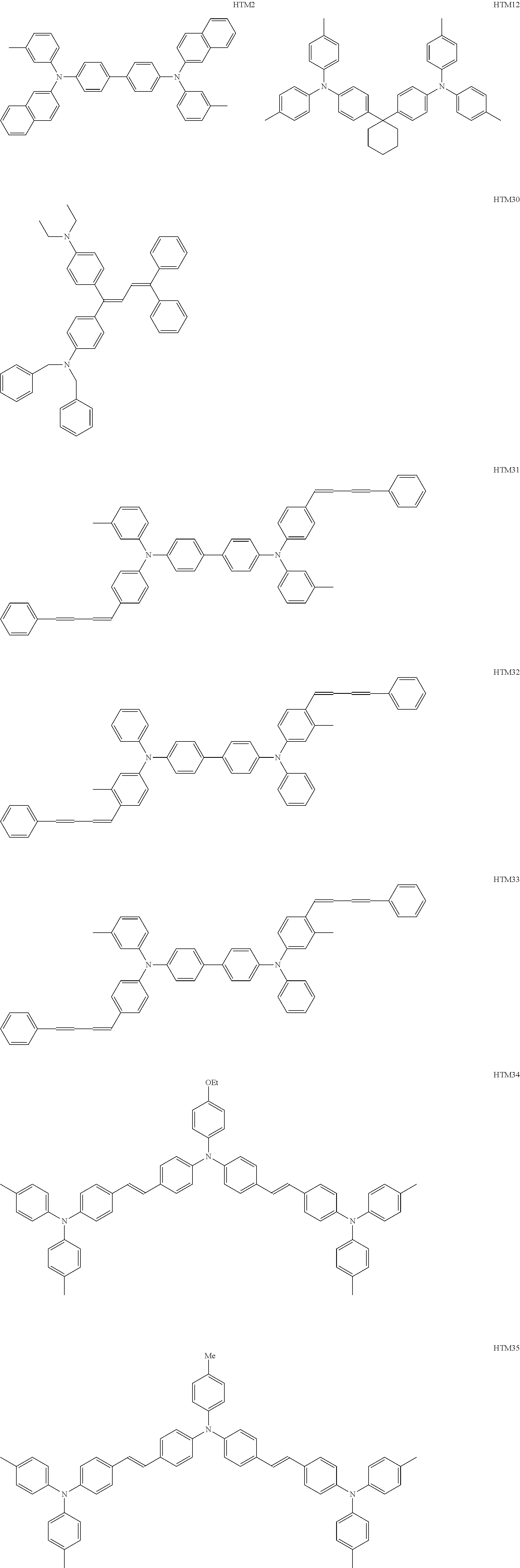

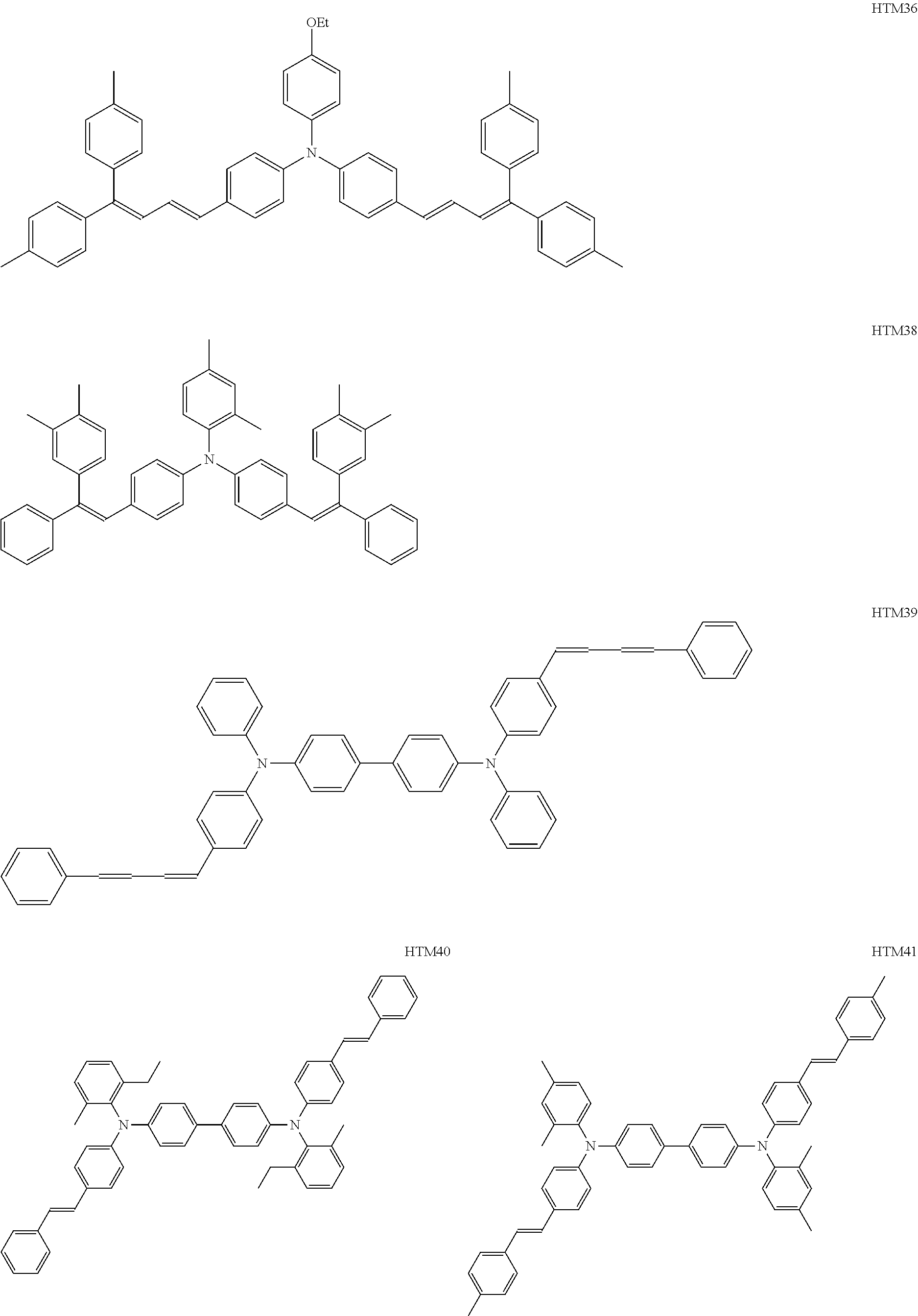

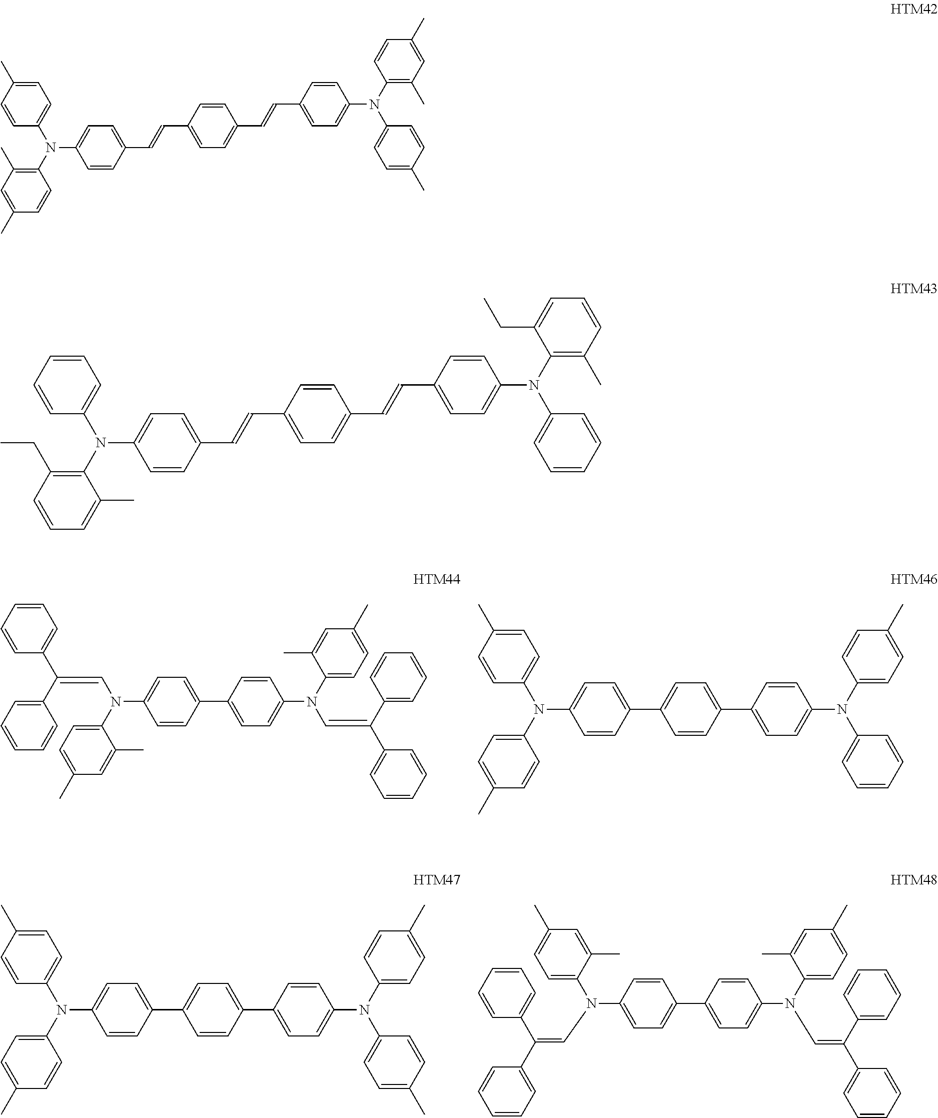

Of the hole transport materials, in terms of the electrical property, HTM31, HTM32, HTM33, HTM34, HTM35, HTM39, HTM40, HTM41, HTM42, HTM43 and HTM48 are preferable, and HTM39, HTM40, HTM41, HTM42, HTM43 and HTM48 are further preferable.

### [Electron Transport Material]

The electron transport material (ETM) can be selected from known materials and used. Examples thereof include electron-withdrawing materials including aromatic nitro compounds such as 2,4,7-trinitrofluorenone, cyano compounds such as tetracyanoquinodimethane, quinone compounds such as diphenoquinone and the like, known cyclic ketone compounds, perylene pigments (perylene derivatives) and the like. Of these, in view of the electrical property, quinone compounds and perylene pigments (perylene derivatives) are preferable, and quinone compounds are more preferable.

Of the quinone compounds, in view of the electrical property, diphenoquinone or dinaphthylquinone is preferable. Of these, dinaphthylquinone is more preferable.

One kind of electron transport material may be used alone, or two or more kinds thereof may be used at any ratio in any combination.

The structures of preferable electron transport materials are exemplified below.

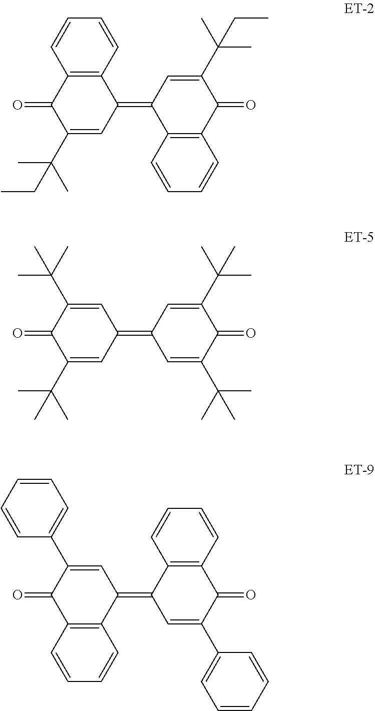 [SMILES: CCC(C)(C)C1=C/C(=C2/C=C(C(C)(C)CC)C(=O)c3ccccc32)c2ccccc2C1=O ; CC(C)(C)C1=CC(=C2C=C(C(C)(C)C)C(=O)C(C(C)(C)C)=C2)C=C(C(C)(C)C)C1=O ; O=C1C(c2ccccc2)=C/C(=C2/C=C(c3ccccc3)C(=O)c3ccccc32)c2ccccc21]

Of the electron transport materials, in terms of the electrical property, ET-2 and ET-5 are preferable, and ET-2 is further preferable.

### (Binder Resin)

Next, the binder resin used for the photosensitive layer of the present invention will be explained.

Examples of the binder resin used for the photosensitive layer of the present invention include: vinyl polymers such as polymethylmethacrylate, polystyrene and polyvinyl chloride or copolymers thereof; vinyl alcohol resins; polyvinyl butyral resins; polyvinyl formal resins; partially modified polyvinyl acetal resins; polyarylate resins; polyamide resins; polyurethane resins; polycarbonate resins; polyester resins; polyester carbonate resins; polyimide resins; phenoxy resins; epoxy resins; silicone resins; and partially cross-linked cured materials thereof. The resins may be modified with a silicon reagent or the like. One kind thereof may be used alone, or two or more kinds thereof can be used at any ratio in any combination.

Moreover, the binder resin used for the photosensitive layer of the present invention preferably contains one kind or two or more kinds of polymer obtained by interfacial polymerization.

The binder resin obtained by interfacial polymerization is preferably a polycarbonate resin or a polyester resin, in particular, preferably a polycarbonate resin or a polyarylate resin. Moreover, in particular, a polymer obtained from an aromatic diol as a raw material is preferable.

### (Other Substances)

In addition to the above materials, the photosensitive layer of the present invention may contain a known additive such as an antioxidant, a plasticizer, an ultraviolet absorber, an electron-withdrawing compound, a leveling agent and a visible light-shielding agent to improve the film forming property, the flexibility, the coatability, the contamination resistance, the gas resistance, the light resistance or the like. Moreover, the photosensitive layer of the present invention may contain various additives such as a sensitizer, a dye, a pigment (excluding those which are the charge generation material, the hole transport material and the electron transport material listed above) and a surfactant according to the need. Examples of the surfactant include silicone oil, a fluorine-based compound and the like. In the present invention, one kind thereof alone or two or more kinds thereof at any ratio in any combination can be appropriately used.

Moreover, for the purpose of reducing the friction resistance on the surface of the photosensitive layer, the photosensitive layer may contain a fluorine-based resin, a silicone resin or the like or may contain particles of such a resin or particles of an inorganic compound such as aluminum oxide.

### (Layer Thickness)

When the photosensitive layer of the present invention is a single-layer photosensitive layer, in view of the dielectric breakdown resistance, the thickness of the photosensitive layer of the present invention is preferably 20 μm or more, in particular, further preferably 25 μm or more. On the other hand, in view of the electrical property, the thickness is preferably 50 μm or less, in particular, further preferably 40 μm or less.

### <Laminate-Type Photosensitive Layer>

When the electrophotographic photoreceptor of the present invention is a laminate-type photosensitive layer, the electrophotographic photoreceptor can have a configuration obtained, for example, by laminating a charge transport layer (CTL) containing a charge transport material on a charge generation layer (CGL) containing a charge generation material (CGM). At this point, a layer other than the charge generation layer (CGL) and the charge transport layer (CTL) can also be included.

### <Charge Generation Layer (CGL)>

The charge generation layer generally contains a charge generation material (CGM) and a binder resin.

The charge generation material (CGM) and the binder resin are the same as those explained for the single-layer photosensitive layer.

### (Other Components)

The charge generation layer can contain another component according to the need in addition to the charge generation material and the binder resin. For example, for the purpose of improving the film forming property, the flexibility, the coatability, the contamination resistance, the gas resistance, the light resistance or the like, a known additive such as an antioxidant, a plasticizer, an ultraviolet absorber, an electron-withdrawing compound, a leveling agent, a visible light-shielding agent and a filler may be contained.

### (Mixing Ratio)

When the proportion of the charge generation material is too high in the charge generation layer, the stability of the coating liquid may decrease due to the cohesion of the charge generation material or the like, while the sensitivity as the photoreceptor may decrease when the proportion of the charge generation material is too low. Thus, regarding the mixing ratio (mass) of the binder resin and the charge generation material, based on 100 parts by mass of the binder resin, the charge generation material is contained preferably at 10 parts by mass or more, in particular, more preferably at 30 parts by mass or more, while the charge generation material is contained preferably at a ratio of 1000 parts by mass or less, in particular, further preferably at a ratio of 500 parts by mass or less, more preferably at a ratio of 300 parts by mass or less in view of the membrane strength, further preferably at a ratio of 200 parts by mass or less.

### (Layer Thickness)

The thickness of the charge generation layer is preferably 0.1 μm or more, in particular, further preferably 0.15 μm or more. On the other hand, the thickness is preferably 10 μm or less, in particular, further preferably 0.6 μm or less.

### <Charge Transport Layer (CTL)>

The charge transport layer (CTL) generally contains a charge transport material and a binder resin. An electron transport material (ETM) may be further contained.

The charge transport material and the binder resin are the same as those explained for the single-layer photosensitive layer.

In the charge transport layer (CTL), regarding the mixing ratio of the binder resin and the hole transport material (HTM), based on 100 parts by mass of the binder resin, the hole transport material (HTM) is preferably blended at a ratio of 20 parts by mass or more, in particular, more preferably at a ratio of 30 parts by mass or more to reduce the residual potential, further preferably at a ratio of 40 parts by mass or more in view of the stability during repeated use and the charge transfer degree. On the other hand, in view of the thermal stability of the photosensitive layer, based on 100 parts by mass of the binder resin, the hole transport material (HTM) is preferably blended at a ratio of 200 parts by mass or less, more preferably at a ratio of 150 parts by mass or less in view of the compatibility of the hole transport material (HTM) and the binder resin, particularly preferably at a ratio of 120 parts by mass or less in view of the glass transition temperature.

### (Other Components)

The charge transport layer can contain another component according to the need in addition to the electron transport material (ETM), the hole transport material (HTM) and the binder resin. For example, for the purpose of improving the film forming property, the flexibility, the coatability, the contamination resistance, the gas resistance, the light resistance or the like, a known additive such as an antioxidant, a plasticizer, an ultraviolet absorber, an electron-withdrawing compound, a leveling agent, a visible light-shielding agent and a filler may be contained.

### (Layer Thickness)

The layer thickness of the charge transport layer is not particularly restricted. In view of the electrical property and the image stability and in view of the high resolution, the thickness is preferably 5 μm or more and 50 μm or less, in particular, more preferably 10 μm or more or 35 μm or less, in particular, further preferably 15 μm or more or 25 μm or less.

### <Formation Method of Photosensitive Layer>

Both for the laminate-type and the single layer, each layer can be formed as follows.

The layers can be formed one by one by repeating coating/drying steps of coating liquids obtained by dissolving or dispersing the materials to be contained in a solvent on a conductive support by a known method such as dip coating, spray coating, nozzle coating, bar coating, roll coating and blade coating.

The forming method, however, is not limited to such a method.

The solvent or the dispersant used for producing the coating liquids is not particularly restricted. Specific examples thereof include an alcohol, an ether, an aromatic hydrocarbon, a chlorinated hydrocarbon and the like. One kind thereof may be used alone, or two or more kinds thereof in any combination of any kinds may be used in combination.

The amount of the solvent or the dispersant used is not particularly restricted. Considering the purposes of the layers and the properties of the solvent/dispersant selected, the amount is preferably appropriately adjusted in such a manner that the solid concentrations of the coating liquids and the physical properties such as the viscosity are in the desired ranges.

The coating films are preferably dried by drying to the touch at room temperature and then heat drying generally in a temperature range of 30° C. or higher and 200° C. or lower for one minute to two hours in still state or with ventilation. The heating temperature may be constant, or heating may be conducted while changing the temperature during drying.

### <Conductive Support of Present Invention>

The conductive support (also referred to as “the conductive support of the present invention”) of the electrophotographic photoreceptor of the present invention is not particularly limited as long as the conductive support supports the layers formed thereon and exhibits conductivity.

As the conductive support of the present invention, for example, a metal material such as aluminum, an aluminum alloy, stainless steel, copper and nickel, a resin material to which conductivity is added by causing conductive powder such as a metal, carbon and tin oxide to coexist, a resin material, glass material or paper material having a surface on which a conductive material such as aluminum, nickel and ITO (indium oxide-tin oxide alloy) is deposited or applied or the like can be mainly used.

Regarding the form of the conductive support of the present invention, a drum, a cylinder, a sheet, a belt or the like is used.

The conductive support of the present invention may be obtained by applying a conductive material having appropriate resistance on a conductive support made of a metal material to regulate the conductivity, the surface property or the like or to cover the defect.

When a metal material such as an aluminum alloy is used as the conductive support of the present invention, the metal material used may be coated with an anodized film.

The average thickness of the anodized film is preferably 20 μm or less, in particular, further preferably 7 μm or less.

When the metal material is coated with an anodized film, sealing treatment is preferably conducted. The sealing treatment can be conducted by a known method.

The surface of the conductive support of the present invention may be smooth or may be roughened using a special cutting method or by subjecting to grinding. Moreover, the surface may be roughened by mixing particles having an appropriate particle size in the material composing the support.

Here, the undercoat layer explained below may be provided between the conductive support of the present invention and the photosensitive layer to improve the adhesion, the blocking property or the like.

### <Undercoat Layer of Present Invention>

The electrophotographic photoreceptor of the present invention may have an undercoat layer (also referred to as “the undercoat layer of the present invention”) between the photosensitive layer of the present invention and the conductive support of the present invention.

As the undercoat layer of the present invention, for example, a resin, a material obtained by dispersing particles of an organic pigment, a metal oxide or the like in a resin or the like can be used.

Examples of the organic pigment used for the undercoat layer of the present invention include a phthalocyanine pigment, an azo pigment, a perylene pigment and the like. Of these, a phthalocyanine pigment or an azo pigment, specifically, the phthalocyanine pigment and the azo pigment used as the charge generation material described above, can be used.

Examples of the metal oxide particles used for the undercoat layer of the present invention include metal oxide particles containing one kind of metal element such as titanium oxide, aluminum oxide, silicon oxide, zirconium oxide, zinc oxide and iron oxide and metal oxide particles containing two or more metal elements such as calcium titanate, strontium titanate and barium titanate. One kind of particles may be used alone for the undercoat layer of the present invention, or more than one kind of particles at any ratio in any combination may be mixed and used.

Of the metal oxide particles, titanium oxide and aluminum oxide are preferable, and in particular, titanium oxide is preferable.

The particle size of the metal oxide particles used for the undercoat layer of the present invention is not particularly limited. Due to the characteristics of the undercoat layer and the stability of the solution for forming the undercoat layer, the average primary particle size is preferably 10 nm or more and is 100 nm or less, more preferably 50 nm or less.

The binder resin used for the undercoat layer of the present invention can be selected, for example, from the following materials and used: a polyvinyl acetal-based resin such as a polyvinyl butyral resin; an insulating resin such as a polyarylate resin, a polycarbonate resin, a polyester resin, a phenoxy resin, an acrylic resin, a methacrylic resin, a polyamide resin, a polyurethane resin, an epoxy resin, a silicone resin, a polyvinyl alcohol resin and a styrene-alkyd resin; and the like. In this regard, however, the binder resin is not limited to these polymers. One kind of the binder resins may be used alone, or two or more kinds thereof may be mixed and used. The binder resin may be used in the form cured with a curing agent.

Of these, a polyvinyl acetal-based resin, an alcohol-soluble copolymerized polyamide, a modified polyamide and the like exhibit excellent dispersibility and coatability and thus are preferable. Of these, an alcohol-soluble copolymerized polyamide is particularly preferable.

The mixing ratio of the particles to the binder resin can be selected freely. It is preferable to use in the range of 10 mass % to 500 mass % in terms of the stability and the coatability of the dispersion.

The thickness of the undercoat layer of the present invention can be selected freely. From the characteristics of the electrophotographic photoreceptor and the coatability of the dispersion, the thickness is preferably 0.1 μm or more and is further preferably 20 μm or less. Moreover, the undercoat layer of the present invention may contain a known antioxidant or the like.

### <Other Layers>

Moreover, the electrophotographic photoreceptor of the present invention may appropriately have another layer according to the need in addition to the conductive support of the present invention, the photosensitive layer of the present invention, the protective layer of the present invention and the undercoat layer of the present invention described above.

### <<Image Formation Device of Present Invention>>

Using the electrophotographic photoreceptor of the present invention, an image formation device (“the image formation device of the present invention”) can be configured.

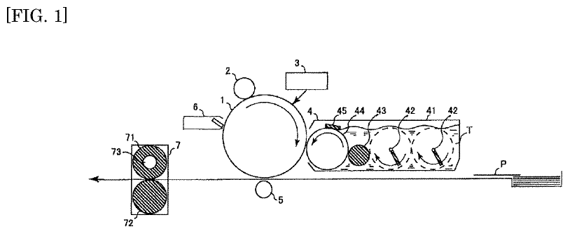

As illustrated in FIG. 1, the image formation device of the present invention is configured with an electrophotographic photoreceptor 1 of the present invention, a charging device 2, an exposure device 3 and a developing device 4, and according to the need, a transfer device 5, a cleaning device 6 and a fixing device 7 are further provided.

The electrophotographic photoreceptor 1 of the present invention is not particularly restricted as long as it is the electrophotographic photoreceptor of the present invention described above. As an example thereof, FIG. 1 illustrates a drum-type photoreceptor in which the photosensitive layer described above is formed on the surface of a cylindrical conductive support. The charging device 2, the exposure device 3, the developing device 4, the transfer device 5 and the cleaning device 6 are arranged along the peripheral surface of the electrophotographic photoreceptor 1 of the present invention.

The charging device 2 can be a non-contact corona charging device such as a corotron and a scorotron or a contact-type charging device (a direct charging device) for charging by bringing a charged material to which voltage is applied into contact with the photoreceptor surface. Examples of the contact charging device include a charging roller, a charging brush and the like. In FIG. 1, a roller-type charging device (charging roller) is illustrated as an example of the charging device 2.

The type of the exposure device 3 is not particularly restricted as long as it can expose the electrophotographic photoreceptor 1 of the present invention and form an electrostatic latent image on the photosensitive surface of the electrophotographic photoreceptor 1 of the present invention.

Moreover, exposure may be conducted by a photoreceptor internal exposure method. The light for the exposure may be any light.

The type of a toner T may be any type, and in addition to a powdery toner, a polymerized toner using a suspension polymerization method, an emulsion polymerization method or the like and the like can be used.

The configuration of the developing device 4 may also be any configuration. The developing device 4 illustrated in FIG. 1 has a configuration in which the toner T is made into a thin layer with a regulating member (a developing blade) 45, charged by friction to predetermined polarity, transferred while supported on a developing roller 44 and brought into contact with the surface of the photoreceptor 1. The configuration, however, is not limited to the configuration.

The type of the transfer device 5 is not particularly restricted, and a device using any type such as an electrostatic image transfer method including corona transfer, roller transfer, belt transfer and the like, a pressure transfer method and an adhesion transfer method can be used.

The cleaning device 6 is not particularly restricted. For example, any cleaning device such as a brush cleaner, a magnetic roller cleaner and a blade cleaner can be used. When a small amount of the toner or almost no toner remains on the surface of the photoreceptor, the cleaning device 6 does not have to be provided.

The fixing device 7 may have any configuration.

Here, the image formation device may have a configuration which can conduct, for example, a charge elimination step in addition to the configuration described above.

Moreover, the image formation device may be configured with further modification and may have, for example, a configuration which can conduct a step such as a pre-exposure step and a supplemental charging step, a configuration for conducting offset printing or a configuration of the full-color tandem type using more than one kind of toner.

### <<Electrophotographic Cartridge of Present Invention>>

The electrophotographic photoreceptor 1 of the present invention can be configured as an integrated cartridge (referred to as “the electrophotographic cartridge of the present invention”) by combining with one or two or more of the charging device 2, the exposure device 3, the developing device 4, the transfer device 5, the cleaning device 6 and the fixing device 7.

The electrophotographic cartridge of the present invention can have a configuration which can be attached to and detached from the main body of an electrophotographic device such as a copier and a laser beam printer. In this case, for example, when the electrophotographic photoreceptor 1 of the present invention or another member is deteriorated, by removing the electrophotographic photoreceptor cartridge from the main body of the image formation device and installing a new electrophotographic photoreceptor cartridge into the main body of the image formation device, maintenance and the management of the image formation device become easy.

### <<Explanation of Terms>>

In the present invention, the expression “X to Y” (X and Y are numbers) includes the meaning of “X or more and Y or less” and the meaning of “preferably larger than X” or “preferably smaller than Y” unless otherwise specified.

Moreover, the expression “X or more” (X is a number) or “Y or less” (Y is a number) also includes the meaning of “preferably larger than X” or “preferably less than Y”.

### EXAMPLES

Embodiments of the present invention will be explained further specifically with Examples below. However, the following Examples are for explaining the present invention in detail, and the present invention is not limited to the Examples shown below and can be carried out with any modification unless departing from the gist thereof. Moreover, the “parts” in the Examples and the Comparative Examples below are “parts by mass” unless otherwise specified.

In the present specification, DMF means N,N-dimethylformamide, and MEHQ means 4-methoxyphenol. Ac means an acetyl group, and 4-DMAP means 4-dimethylaminopyridine.

### <Synthesis of Electron-Transporting Compound>

Next, synthetic methods of a compound 1, a compound 2, a comparative compound 2 and a comparative compound 3 as electron-transporting compounds will be explained. Here, as the comparative compound 1, a compound having the structure shown below was used.

### [Synthesis of Compound 1]

The synthesis scheme of the compound 1 is shown below.

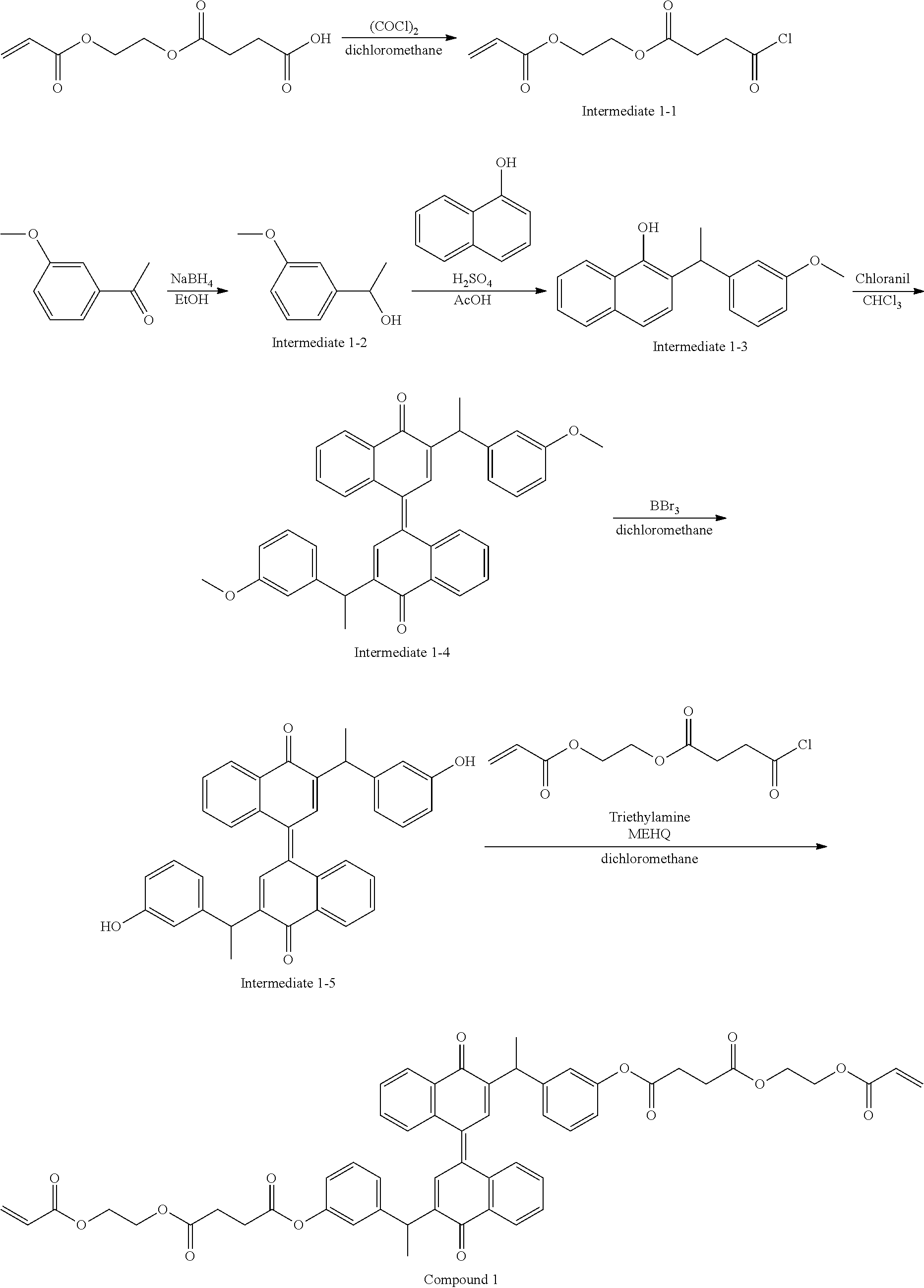

The synthesis procedures of the compound 1 are shown below.

### (Synthesis of Intermediate 1-1)

In nitrogen atmosphere, a solution was prepared by adding 100 mL of dichloromethane to mono(2-acryloyloxyethyl) succinate (11.7 g, 54.1 mmol) and ice-cooled. Oxalyl chloride (6.0 mL, 70.3 mmol) was added dropwise to the solution, and the mixture was stirred under ice-cooling for an hour and stirred at room temperature for 12 hours. After evaporating the solvent at reduced pressure, an intermediate 1-1 (an amount of 12 g, a yield of 94%) was obtained.

### (Synthesis of Intermediate 1-2)

In nitrogen atmosphere, 300 mL of ethanol was added to 3-methoxyacetophenone (16 g, 106.5 mmol), and the mixture was ice-cooled. Sodium borohydride (6.0 g, 159.8 mmol) in several portions was added, and the mixture was stirred under ice-cooling for an hour and stirred at room temperature for two hours. The reaction solution was poured into 300 ml of water. The organic layer was subjected to extraction with ethyl acetate, and the organic layer was washed with salt water. After adding magnesium sulfate and drying, the solvent was evaporated at reduced pressure, and an intermediate 1-2 (an amount of 15.1 g, a yield of 94%) was obtained.

### (Synthesis of Intermediate 1-3)

In nitrogen atmosphere, 300 mL of acetic acid was added to a mixture of 1-naphthol (32.5 g, 225.4 mmol) and the intermediate 1-2 (22.9 g, 150.2 mmol), and 8.0 mL of sulfuric acid was added dropwise. After stirring at room temperature for 40 minutes, the mixture was poured into 300 mL of water. The organic layer was subjected to extraction with dichloromethane, and the organic layer was washed with water. After adding magnesium sulfate and drying, the solvent was evaporated at reduced pressure, and the residue was subjected to silica gel column chromatography. Thus, an intermediate 1-3 (an amount of 17 g, a yield of 41%) was obtained.

### (Synthesis of Intermediate 1-4)

In nitrogen atmosphere, 500 mL of chloroform was added to the intermediate 1-3 (17 g, 61.1 mmol), and chloranil (15 g, 61.1 mmol) was added. After stirring at room temperature for 24 hours, the mixture was filtered with dichloromethane. The solvent of the filtrate was evaporated at reduced pressure, and the residue was subjected to silica gel chromatography. Thus, an intermediate 1-4 (an amount of 15 g, a yield of 89%) was obtained.

### (Synthesis of Intermediate 1-5)

In nitrogen atmosphere, 200 mL of dichloromethane was added to the intermediate 1-4 (14 g, 25.3 mmol), and the mixture was cooled to −70° C. A dichloromethane solution of 1 mol/L boron tribromide in an amount of 130 mL was added dropwise, and the mixture was heated to room temperature and stirred for 12 hours. After cooling to 0° C., 150 mL of water was added dropwise. After heating to room temperature, the organic layer was subjected to extraction with dichloromethane, and the organic layer was washed with water. After adding magnesium sulfate and drying, the resultant was dissolved in 200 mL of chloroform, and chloranil (5.1 g, 20.7 mmol) was added. The mixture was stirred at room temperature for 2.5 hours. The mixture was filtered with dichloromethane. The solvent of the filtrate was evaporated at reduced pressure, and the residue was subjected to silica gel chromatography. Thus, an intermediate 1-5 (an amount of 9.6 g, a yield of 72%) was obtained.

### (Synthesis of Compound 1)

In nitrogen atmosphere, a solution was prepared by adding 100 mL of dichloromethane to a mixture of the intermediate 1-5 (7.6 g, 14.5 mmol) and 0.05 g of 4-methoxyphenol and ice-cooled. After adding triethylamine (8.0 mL, 58.0 mmol) to the solution, the intermediate 1-1 (7.5 g, 31.9 mmol) was added dropwise, and the mixture was stirred under ice-cooling for an hour and stirred at room temperature for 12 hours. The reaction solution was poured into 200 ml of water, and extraction with dichloromethane was conducted. The organic layer was washed with water. After adding magnesium sulfate and drying, the solvent was evaporated at reduced pressure, and the residue was subjected to silica gel chromatography. Thus, the compound 1 (an amount of 4.4 g, a yield of 33%) was obtained.

[Synthesis of Compound 2]

The synthesis scheme of the compound 2 is shown below.

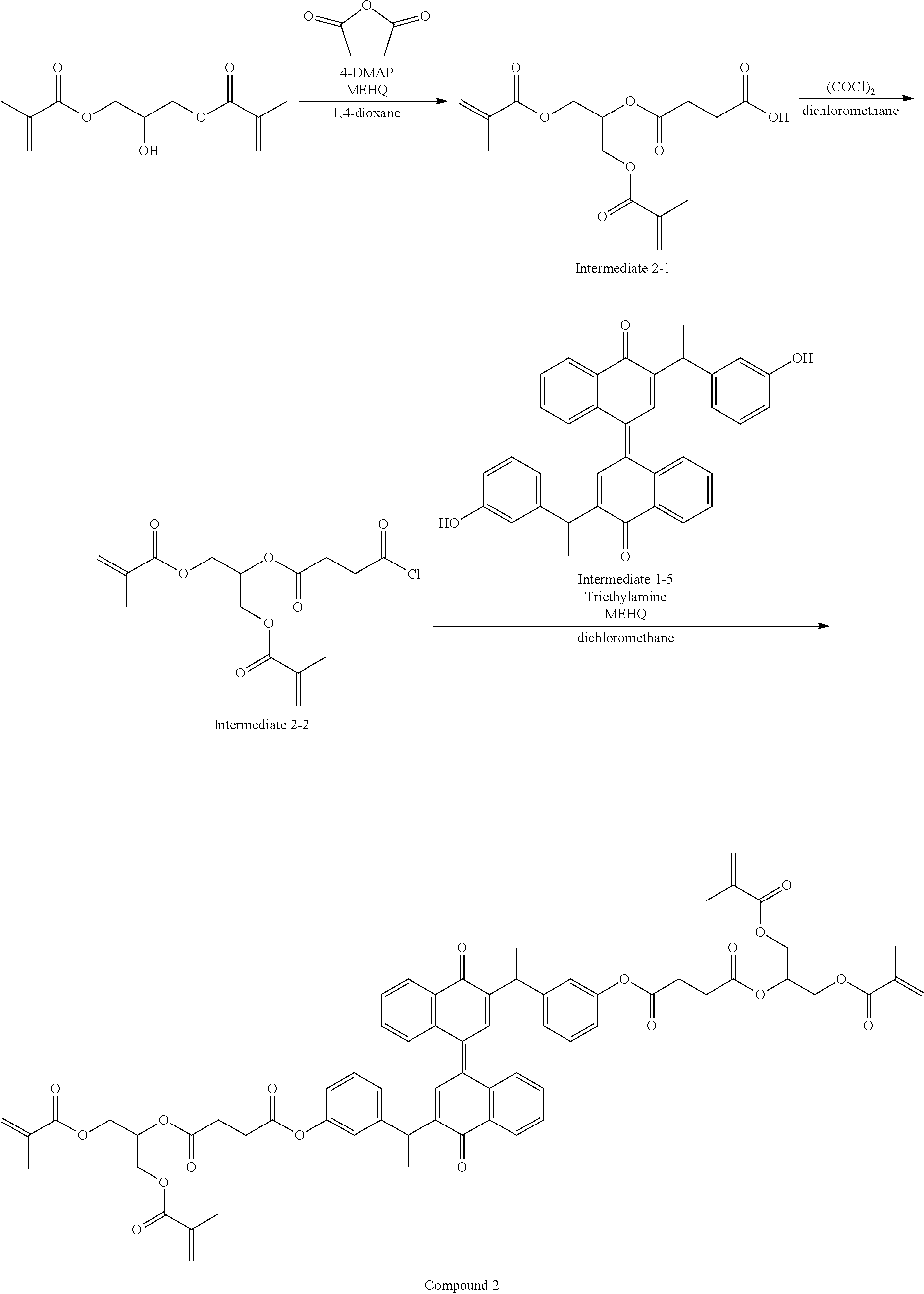

The synthesis procedures of the compound 2 are shown below.

### (Synthesis of Intermediate 2-1)

In nitrogen atmosphere, a solution was prepared by adding 100 mL of 1,4-dioxane to a mixture of succinic anhydride (11.0 g, 109.5 mmol) and 4-DMAP (0.26 g, 2.19 mmol). A solution of glycerol dimethacrylate (25 g, 109.5 mmol) and MEHQ (27 mg, 0.22 mmol) dissolved in 50 mL of 1,4-dioxane was added dropwise to the solution, and the mixture was stirred at 80° C. for nine hours. After cooling to room temperature, the mixture was poured into 200 mL of water, and extraction with dichloromethane was conducted. After the organic layer was washed with water, magnesium sulfate was added to dry. The solid was filtered, and the solvent of the filtrate was evaporated at reduced pressure. After drying the residue, an intermediate 2-1 (an amount of 30 g, a yield of 83%) was obtained.

### (Synthesis of Intermediate 2-2)

In nitrogen atmosphere, 100 mL of dehydrated dichloromethane and 1 mL of dehydrated dimethylformamide were added to the intermediate 2-1 (21.6 g, 65.8 mmol), and the mixture was ice-cooled. Oxalyl chloride (11.2 mL, 131.6 mmol) was added dropwise, and the mixture was stirred under ice-cooling for two hours and stirred at room temperature for 12 hours. After evaporating the solvent at reduced pressure, the residue was dried, and an intermediate 2-2 (an amount of 21.5 g, a yield of 94%) was obtained.

### (Synthesis of Compound 2)

In nitrogen atmosphere, a solution was prepared by adding 200 mL of dichloromethane to a mixture of the intermediate 1-5 (5.5 g, 10.5 mmol) obtained by the procedures described above and 0.05 g of MEHQ and ice-cooled. After adding triethylamine (7.3 mL, 52.4 mmol) to the solution, the intermediate 2-2 (10.9 g, 31.5 mmol) was added dropwise, and the mixture was stirred under ice-cooling for an hour and at room temperature for two hours. The reaction solution was poured into 100 mL of water, and extraction with dichloromethane was conducted. The organic layer was washed with water. After adding magnesium sulfate and drying, the solvent was evaporated at reduced pressure, and the residue was subjected to silica gel chromatography. Thus, the compound 2 (an amount of 4.8 g, a yield of 40%) was obtained.

[Synthesis of Comparative Compound 2]

The synthesis scheme of the comparative compound 2 is shown below.

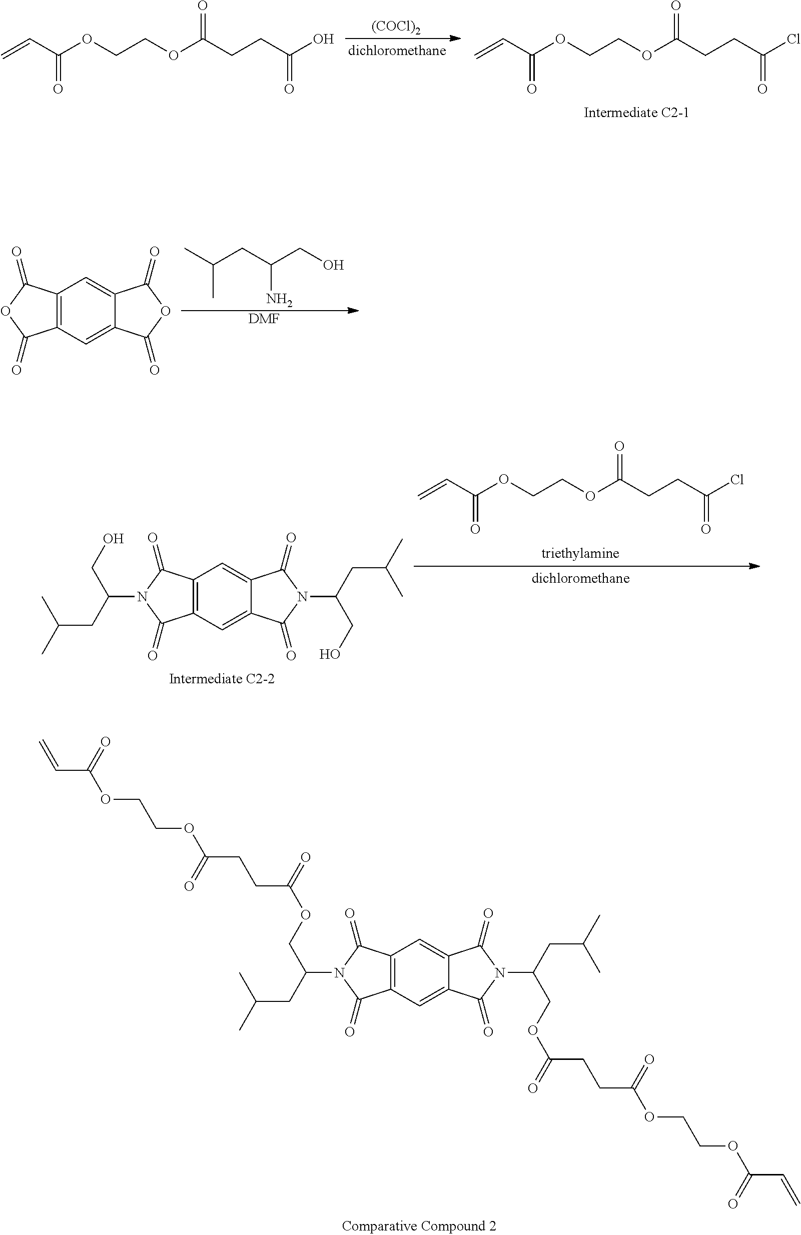

The synthesis procedures of the comparative compound 2 are shown below.

### (Synthesis of Intermediate C2-1)

In nitrogen atmosphere, 200 mL of dehydrated dichloromethane was added to mono(2-acryloyloxyethyl) succinate (21.7 g, 100.4 mmol), and the mixture was ice-cooled. Oxalyl chloride (11.2 mL, 130.5 mmol) was added dropwise, and the mixture was stirred under ice-cooling for an hour and stirred at room temperature for 12 hours. The solvent in the reaction solution was evaporated at reduced pressure, and the residue was dried. Thus, a compound C2-1 (an amount of 23 g, a yield of 97%) was obtained.

### (Synthesis of Intermediate C2-2)

In nitrogen atmosphere, 100 mL of N,N-dimethylformamide was added to a mixture of benzene-1,2,4,5-tetracarboxylic anhydride (5.0 g, 22.9 mmol) and L-(+)-leucinol (5.9 mL, 45.8 mmol), and the mixture was stirred at 120° C. for nine hours. After cooling to room temperature, the mixture was poured into 200 mL of ice water, and the solution was made acidic by adding 1 N hydrochloric acid. The solid was filtered and washed with water. After drying, an intermediate C2-2 (an amount of 8.2 g, a yield of 86%) was obtained.

### (Synthesis of Comparative Compound 2)

In nitrogen atmosphere, 100 mL of dehydrated dichloromethane and triethylamine (7.0 mL, 50.4 mmol) were added to the intermediate C2-2 (5.2 g, 12.6 mmol), and the mixture was ice-cooled. The intermediate C2-1 (6.5 g, 27.7 mmol) which was dissolved in 100 mL of dehydrated dichloromethane was added dropwise, and the mixture was stirred under ice-cooling for an hour and stirred at room temperature for an hour. The reaction solution was poured into 100 mL of water, and extraction with dichloromethane was conducted. After the organic layer was washed with water, magnesium sulfate was added to dry. The solid was filtered, and the solvent of the filtrate was evaporated at reduced pressure. The residue was subjected to silica gel column chromatography, and the comparative compound 2 (an amount of 7.6 g, a yield of 75%) was obtained.

### [Synthesis of Comparative Compound 3]

The synthesis scheme of the comparative compound 3 is shown below.

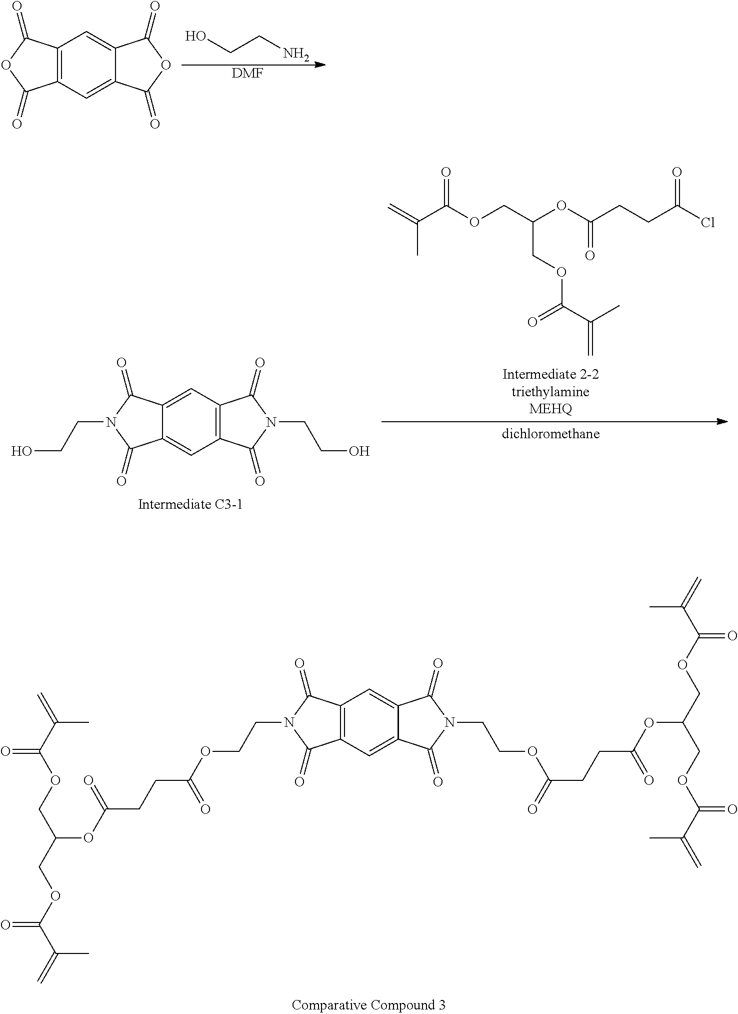

### (Synthesis of Intermediate C3-1)

In nitrogen atmosphere, 80 mL of N,N-dimethylformamide was added to a mixture of benzene-1,2,4,5-tetracarboxylic anhydride (5.3 g, 24.3 mmol) and 2-aminoethanol (3.0 mL, 48.6 mmol), and the mixture was stirred at 120° C. for five hours. After cooling to room temperature, the mixture was poured into 100 mL of ice water, and the solution was made acidic by adding 1 N hydrochloric acid. The solid was filtered and washed with water. After drying, an intermediate C3-1 (an amount of 3.9 g, a yield of 53%) was obtained.

### (Synthesis of Comparative Compound 3)

In nitrogen atmosphere, a solution was prepared by adding 70 mL of dichloromethane to a mixture of the intermediate C3-1 (3.0 g, 9.93 mmol) and 0.05 g of MEHQ and ice-cooled. After adding triethylamine (6.9 mL, 49.7 mmol) to the solution, a solution obtained by dissolving the intermediate 2-2 (10.3 g, 29.8 mmol) obtained by the procedures described above in 30 mL of dichloromethane was added dropwise, and the mixture was stirred under ice-cooling for an hour and stirred at room temperature for 12 hours. The reaction solution was poured into 100 mL of water, and extraction with dichloromethane was conducted. The organic layer was washed with water. After adding magnesium sulfate and drying, the solvent was evaporated at reduced pressure, and the residue was subjected to silica gel chromatography. Thus, the comparative compound 3 (an amount of 5.2 g, a yield of 57%) was obtained.

### <<Evaluation of Solubility in Organic Solvent>>

### Examples 1-1 and 1-2 and Comparative Example 1-1

To 100 parts by mass of a mixed solvent of tetrahydrofuran (appropriately abbreviated to as THF below), toluene (appropriately abbreviated to as TL below) and 2-propanol (appropriately abbreviated to as 2-PrOH below) (19 mass % THF, 38 mass % TL and 43 mass % 2-PrOH, 25° C.), eight parts by mass of the compound 1 or 2 or the comparative compound 1 was added, and without heating or cooling, the mixtures were stirred with a rotor for 10 minutes. Then, the dissolution states were observed visually, and the solubility was evaluated by the following criteria. The evaluation results are shown in Table 1. Here, a compound having the structure shown below was used as the comparative compound 1.
A (good): The compound dissolved completely at room temperature.
B (poor): Undissolved compound was observed at room temperature.

TABLE 1

| | Electron-Transporting | |
|---|---|---|
| | Compound | Solubility |
| Example 1-1 | Compound 1 | A |
| Example 1-2 | Compound 2 | A |
| Comparative | Comparative | B |
| Example 1-1 | Compound 1 | |

### <Discussion>

From the evaluation of the solubility, the results of the tests which were conducted so far by the present inventors and the like, it was found that the novel compound represented by the formula (1) below has electron-transporting property and also dissolves sufficiently in an organic solvent. It is believed that, because the compound represented by the formula (1) below has a structure in which a chain-polymerizable functional group is bonded to a dinaphthoquinone skeleton having electron-transporting property through an aromatic group, namely through an aromatic group as a spacer, the amorphousness improves, and the solubility in an organic solvent improves. Moreover, it was found that the novel compound represented by the formula (1) below is useful as an electron-transporting compound of an electrophotographic photoreceptor.

As a result, it was found that addition of an electron-transporting compound having the structure represented by the formula (1) below, namely a structure in which a dinaphthoquinone skeleton is bonded to a chain-polymerizable functional group through an aromatic group, namely through an aromatic group as a spacer, to the protective layer can add electron-transporting property to the protective layer, achieve an excellent electrical property, in particular excellent residual potential property, suppress aggregation or precipitation of the electron-transporting compound and provide a uniform protective layer (film) even when a compound having an electron-transporting structure is contained in the protective layer.

As a factor which can achieve an excellent electrical property, it is believed that excellent electron-transporting property is exhibited because the dinaphthoquinone skeleton has π-electron conjugated system and is a skeleton with planarity and because the electron affinity is thus high. Moreover, as a factor enabling formation of a uniform protective layer, it is believed that a uniform protective layer can be formed because the amorphousness of the electron-transporting compound improves and because the solubility of the electron-transporting compound in the coating liquid for forming the protective layer improves when the dinaphthoquinone skeleton is bonded to a chain-polymerizable functional group through an aromatic group, namely through an aromatic group as a spacer. Compared to an electron-transporting compound having a structure in which a dinaphthoquinone skeleton is bonded to a chain-polymerizable functional group through an alkyl group as a spacer, when an aromatic group is a spacer, the steric hindrance is caused more, resulting in suppression of aggregation of the molecules, further improvement of the amorphousness of the electron-transporting compound and further improvement of the solubility of the electron-transporting compound in the coating liquid for forming the protective layer.

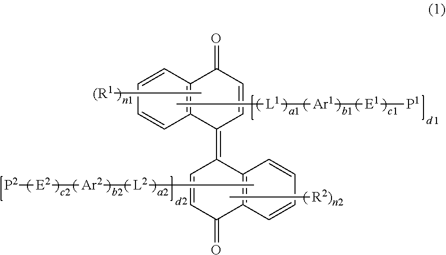

(In the formula (1), L¹ and L² each independently represent a divalent group. Ar¹ and Ar² each independently represent a divalent aromatic group having 6 to 30 carbon atoms or a divalent heteroaromatic group having 3 to 30 carbon atoms. E¹ and E² each independently represent a divalent group. P¹ and P² each independently represent a chain-polymerizable functional group. R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents. a1, a2, b1, b2, c1 and c2 each independently represent an integer of 1 or more. d1 and d2 each independently represent an integer of 0 or more and 6 or less. Here, d1+d2 is one or more. n1 and n2 each independently represent an integer of 0 or more.)

### <<Production of Electrophotographic Photoreceptor>>

### (Production of Coating Liquid P¹ for Forming Undercoat Layer)

Twenty parts of D-form titanyl phthalocyanine, which exhibits a clear peak at a diffraction angle of 2θ=27.3°±0.2° in powder X-ray diffraction using CuKα rays, and 280 parts of 1,2-dimethoxyethane were mixed, and the mixture was ground with a sand grind mill for two hours to conduct atomization dispersion treatment. Furthermore, 400 parts of 1,2-dimethoxyethane solution containing 2.5 mass % polyvinyl butyral (manufactured by Denki Kagaku Kogyo Kabushiki Kaisha, product name “Denka Butyral” #6000C) and 170 parts of 1,2-dimethoxyethane were added thereto and mixed, and a coating liquid P¹ for forming an undercoat layer having a solid concentration of 3.4 mass % was produced.

### (Production of Coating Liquid Q1 for Forming Single-Layer Photosensitive Layer)

To 793.35 parts of a mixed solvent of tetrahydrofuran (appropriately abbreviated to as THE below) and toluene (appropriately abbreviated to as TL below) (80 mass % THF and 20 mass % TL), 2.6 parts of D-form titanyl phthalocyanine, which exhibits a clear peak at a diffraction angle of 2θ=27.3°±0.2° in powder X-ray diffraction using CuKα rays, 1.3 parts of a perylene pigment 1 having the structure below, 0.5 parts of a polyvinyl butyral resin, 100 parts of the hole transport material below (HTM48, molecular weight of 748), 60 parts of the electron transport material below (ET-2, molecular weight of 424.2), 100 parts of a polycarbonate resin having a biphenyl structure and 0.05 parts of silicone oil (manufactured by Shin-Etsu Chemical Co., Ltd.: product name KF-96) as a leveling agent were added and mixed, and a coating liquid Q1 for forming a single-layer photosensitive layer having a solid concentration of 25 mass % was produced.

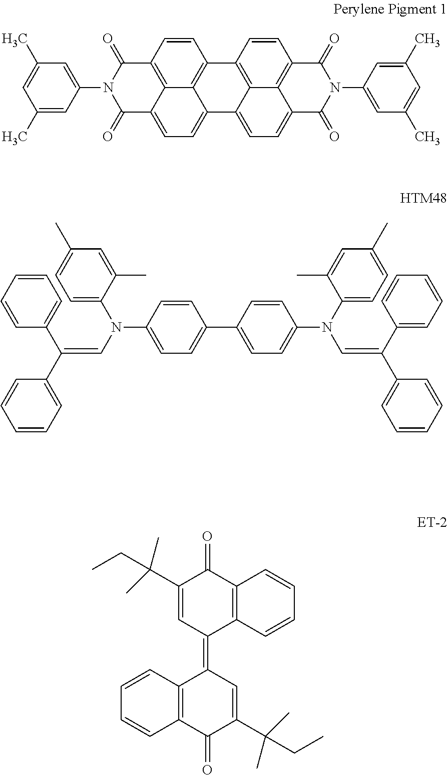

### (Production of Coating Liquid S1 for Forming Protective Layer)

The compound 1 below as an electron-transporting compound and benzophenone and Omnirad TPO H (2,4,6-trimethylbenzoyl-diphenylphosphine oxide) as polymerization initiators were weighed in such a manner that the mass ratio became compound 1/benzophenone/Omnirad TPO H=100/1/2, and these materials were dissolved in a mixed solvent of methanol/1-propanol (methanol/1-propanol=(mass ratio) 8/2). Thus, a coating liquid S1 for forming a protective layer (solid concentration of 8.0 mass %) was obtained.

### [Compound 1]

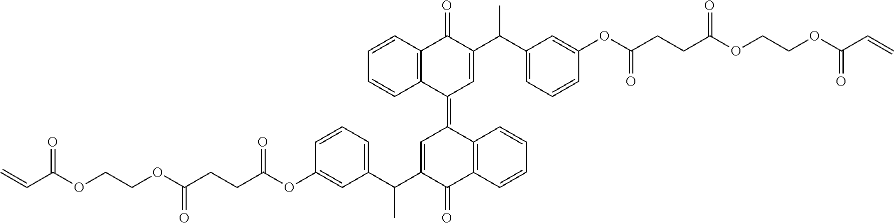 [SMILES: C=CC(=O)OCCOC(=O)CCC(=O)Oc1cccc(C(C)C2=C/C(=C3/C=C(C(C)c4cccc(OC(=O)CCC(=O)OCCOC(=O)C=C)c4)C(=O)c4ccccc43)c3ccccc3C2=O)c1]

### (Production of Coating Liquid S2 for Forming Protective Layer)

A curable compound having the structure below (polyester acrylate: manufactured by Toagosei Co., Ltd., product name “Aronix M-9050”), the compound 1 and benzophenone and Omnirad TPO H (2,4,6-trimethylbenzoyl-diphenylphosphine oxide) as polymerization initiators were weighed in such a manner that the mass ratio became compound 1/curable compound (M-9050)/benzophenone/Omnirad TPO H=100/50/1/2, and these materials were dissolved in a mixed solvent of methanol/1-propanol (methanol/1-propanol=(mass ratio) 8/2). Thus, a coating liquid S2 for forming a protective layer (solid concentration of 8.0 mass %) was obtained.

### (Production of Coating Liquids S3 to S7 for Forming Protective Layer)

Coating liquids S3 to S7 for forming a protective layer were obtained in the same manner as the coating liquid S1 for forming a protective layer except that the kind of the electron-transporting compound and the amount of the curable compound (M-9050) were changed as shown in Table 2.

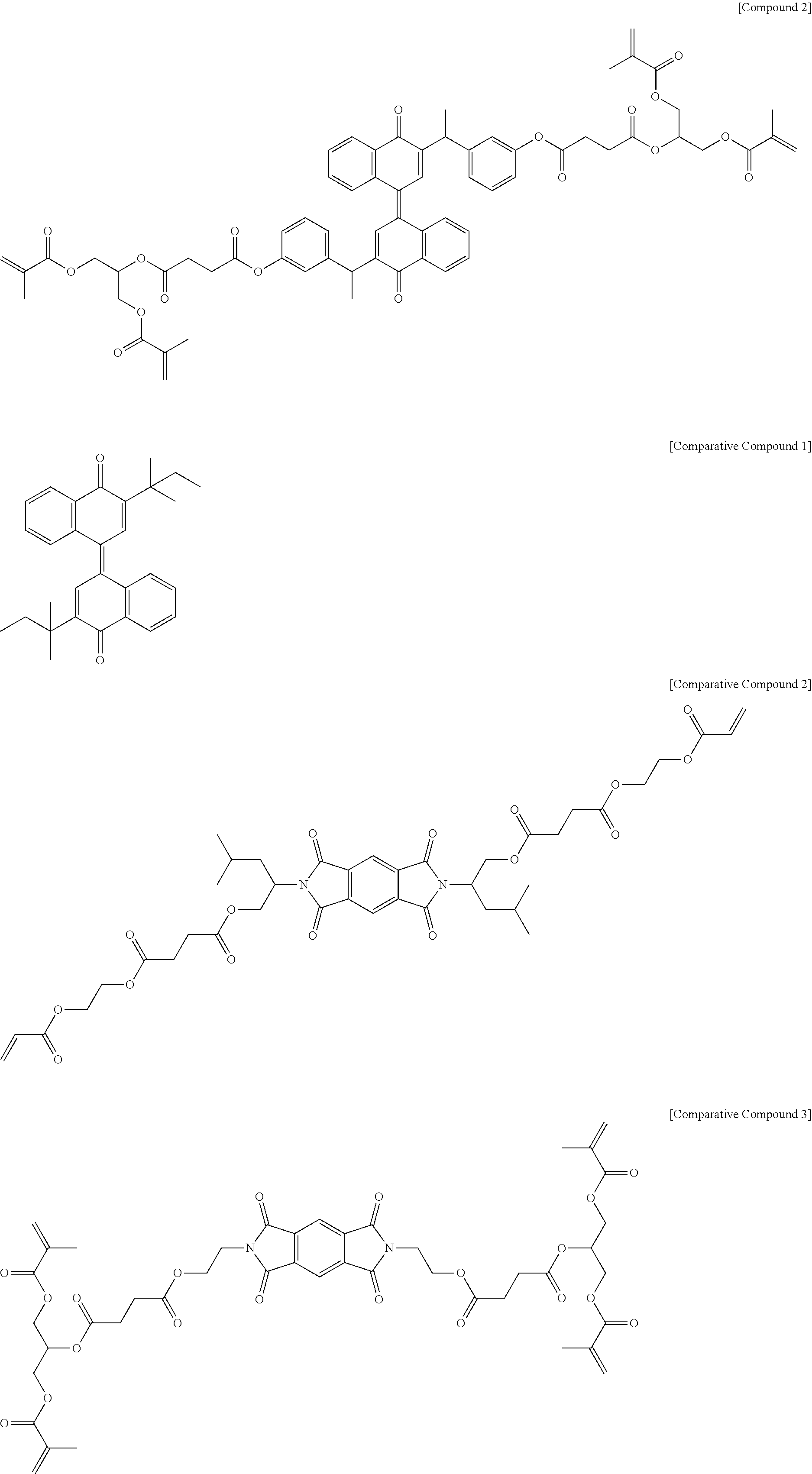

### <Production of Single-Layer Photoreceptor>

Single-layer photoreceptors were produced by the procedures below.

### Example 2-1

An aluminum cylinder of 30 mmφ and a length of 244 mm having a surface subjected to cutting treatment was coated with the coating liquid P¹ for forming an undercoat layer by dip coating, and thus an undercoat layer was provided in such a manner that the thickness after drying became 0.3 μm. The coating liquid Q1 for forming a single-layer photosensitive layer was applied on the undercoat layer by dip coating and dried at 125° C. for 24 minutes, and thus a single-layer photosensitive layer was provided in such a manner that the thickness after drying became 32 μm. The coating liquid S1 for forming a protective layer was applied on the single-layer photosensitive layer by ring coating, and soon after the application, LED light of 365 nm was applied at an intensity of 108 J/cm² while the photoreceptor was rotated at 60 rpm in nitrogen atmosphere to provide a protective layer in such a manner that the thickness after curing became 1 μm. Thus, a photoreceptor A1 was produced.

### Examples 2-2 to 2-4 and Comparative Examples 2-1 to 2-3

Photoreceptors A2 to A7 were produced in the same manner as the photoreceptor A1 except that the coating liquid S1 for forming a protective layer was changed to the coating liquids S2 to S7 for forming a protective layer.

### <Presence or Absence of Aggregates/Precipitates in Protective Layer>

The photoreceptors A1 to A7 obtained in the Examples and the Comparative Examples were visually observed and evaluated by the following criteria.

The photoreceptor was evaluated as unacceptable “B” when aggregates or precipitates were observed in the protective layer, and the photoreceptor was evaluated as acceptable “A” when no aggregates or no precipitates were observed in the protective layer.

### <Electrical Property: Evaluation of Residual Potential>

The photoreceptors A1 to A7 obtained in the Examples and the Comparative Examples were attached to a device for evaluating electrophotographic properties produced according to the measurement standards of the Society of Electrophotography (described in Sequel to Basics and Application of Electrophotographic Technology, edited by the Society of Electrophotography, Corona Publishing Co., Ltd., pages 404 to 405), and the electrical properties after charging, exposure, potential measurement and charge elimination cycles were measured as follows.

First, the grid voltage was adjusted, and the photoreceptor was charged to an initial surface potential (V0) of +700 V. Next, 1.3 μJ/cm² of exposure light was applied, and the residual potential (VL) 60 milliseconds after the application was measured. Here, as the exposure light, monochromatic light of 780 nm from the light of a halogen lamp through an interference filter was used. The measurement environment was at a temperature of 25° C. and at a relative humidity of 50% (N/N environment).

The residual potentials (VL) are shown in Table 2. As the absolute value of the residual potential (VL) is smaller, the potential lowers because the charge is sufficiently transported, which is considered a good result.

In the present invention, the residual potential (VL) of 255 V or less was evaluated as “acceptable”.

The solubility of the photoreceptor A5 in methanol/1-propanol was poor, and the membrane uniformity of the protective layer was poor. Thus, the residual potential could not be evaluated.

TABLE 2

| | | Protective Layer | | | | |
|---|---|---|---|---|---|---|
| | | Coating | | | | |
| | | Liquid for | | | | |
| | Photo- | Forming | | | Presence or Absence of | Residual |
| | recep- | Protective | Electron-Transporting Compound | Curable Compound | Aggregates/Precipitates | Potential |
| | tor | Layer | (parts by mass) | (parts by mass) | in Protective Layer | VL (V) |
| Example 2-1 | A 1 | S 1 | Compound 1 (100) | M-9050 (0) | A | 160 |
| Example 2-2 | A 2 | S 2 | Compound 1 (100) | M-9050 (50) | A | 209 |
| Example 2-3 | A 3 | S 3 | Compound 1 (100) | M-9050 (100) | A | 234 |
| Example 2-4 | A 4 | S 4 | Compound 2 (100) | M-9050 (0) | A | 250 |
| Comparative Example 2-1 | A 5 | S 5 | Comparative Compound 1 (100) | M-9050 (100) | B | Not Evaluable |
| Comparative Example 2-2 | A 6 | S 6 | Comparative Compound 2 (100) | M-9050 (100) | A | 260 |
| Comparative Example 2-3 | A 7 | S 7 | Comparative Compound 3 (100) | M-9050 (100) | A | 330 |

### <Discussion>

From the results in Table 2, in all of Examples 2-1 to 2-4, compared to Comparative Examples 2-1 to 2-3, there were no aggregates/precipitates in the protective layers, and protective layers having a uniform composition could be obtained. Moreover, a superior effect regarding the residual potential property was observed.

As a result, it was found that, in an electrophotographic photoreceptor having at least a photosensitive layer and a protective layer in this order on a conductive support, addition of an electron-transporting compound having the structure represented by the formula (1) below, namely a structure in which a dinaphthoquinone skeleton is bonded to a chain-polymerizable functional group through an aromatic group, namely through an aromatic group as a spacer, to the protective layer can add electron transporting property to the protective layer, achieve an excellent electrical property, in particular excellent residual potential property, suppress aggregation or precipitation of the electron-transporting compound and provide a protective layer (film) having a uniform composition even when a compound having an electron-transporting structure is contained in the protective layer.

As a factor which can achieve an excellent electrical property, it is believed that excellent electron-transporting property is exhibited because the dinaphthoquinone skeleton has n electron conjugated system and is a skeleton with planarity and because the electron affinity is thus high. Moreover, as a factor enabling formation of a protective layer having a uniform composition, it is believed that a protective layer having a uniform composition can be formed because the amorphousness of the electron-transporting compound improves and because the solubility of the electron transporting compound in the coating liquid for forming the protective layer improves when the dinaphthoquinone skeleton is bonded to a chain-polymerizable functional group through an aromatic group, namely through an aromatic group as a spacer. Compared to an electron-transporting compound having a structure in which a dinaphthoquinone skeleton is bonded to a chain-polymerizable functional group through an alkyl group as a spacer, when an aromatic group is a spacer, the steric hindrance is caused more, resulting in suppression of aggregation of the molecules, further improvement of the amorphousness of the electron-transporting compound and further improvement of the solubility of the electron-transporting compound in the coating liquid for forming the protective layer.

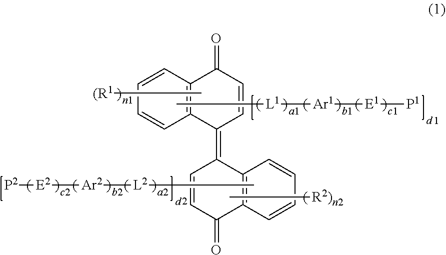

(In the formula (1), L¹ and L² each independently represent a divalent group. Ar¹ and Ar² each independently represent a divalent aromatic group having 6 to 30 carbon atoms or a divalent heteroaromatic group having 3 to 30 carbon atoms. E¹ and E² each independently represent a divalent group. P¹ and P² each independently represent a chain polymerizable functional group. R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents. a1, a2, b1, b2, c1 and c2 each independently represent an integer of 1 or more. d1 and d2 each independently represent an integer of 0 or more and 6 or less. Here, d1+d2 is one or more. n1 and n2 each independently represent an integer of 0 or more.)

### REFERENCE SIGNS LIST

1: Photoreceptor (electrophotographic photoreceptor)
2: Charging device (charging roller; charging member)
3: Exposure device (exposure member)
4: Developing device (developing member)
5: Transfer device
6: Cleaning device
7: Fixing device
41: Developer tank
42: Agitator
43: Supply roller
44: Developing roller
45: Regulating member
71: Upper fixing member (pressure roller)
72: Lower fixing member (fixing roller)
73: Heating device
T: Toner
P: Recording sheet (paper, medium)

1. An electrophotographic photoreceptor comprising a photosensitive layer and a protective layer in this order on a conductive support,
wherein the protective layer comprises a polymer of an electron-transporting compound represented by the following formula (1): 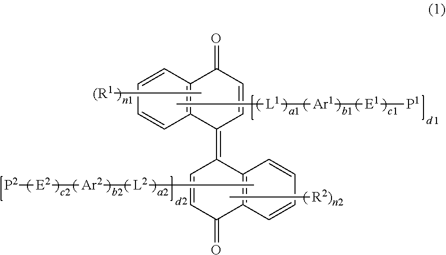
in formula (1),
L¹ and L² each independently represent a divalent group,
Ar¹ and Ar² each independently represent a divalent aromatic group having 6 to 30 carbon atoms or a divalent heteroaromatic group having 3 to 30 carbon atoms,
E¹ and E² each independently represent a divalent group,
P¹ and P² each independently represent a chain polymerizable functional group,
R¹ and R² each independently represent an alkyl group which may have one or more substituents one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents,
a1, a2, b1, b2, c1 and c2 each independently represent an integer of 1 or more,
d1 and d2 each independently represent an integer of 0 or more and 6 or less, wherein d1+d2 is one or more, and n1 and n2 each independently represent an integer of 0 or more.

2. The electrophotographic photoreceptor according to claim 1, wherein P¹ and P² in formula (1) are each independently an acryloyl group or a methacryloyl group.

3. The electrophotographic photoreceptor according to claim 1, wherein Ar¹ and Ar² in formula (1) are each independently a phenylene group, a naphthylene group or a pyridylene group.

4. The electrophotographic photoreceptor according to claim 1, wherein L¹ and L² in formula (1) are each independently an alkylene group.

5. The electrophotographic photoreceptor according to claim 1, wherein E¹ and E² in formula (1) are each independently a divalent group having an ester bond.

6. The electrophotographic photoreceptor according to claim 5, wherein E¹ and E² in formula (1) are each independently a divalent group having two or more ester bonds.

7. The electrophotographic photoreceptor according to claim 6, wherein the divalent group having two or more ester bonds comprises a structure represented by formula (E-1) or formula (E-2)

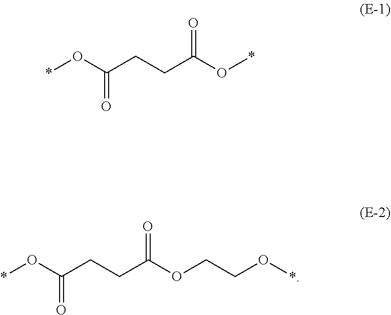

8. The electrophotographic photoreceptor according to claim 1, wherein the protective layer comprises 40 parts by mass or more of the electron-transporting compound based on a total mass of 100 parts by mass of the protective layer.

9. The electrophotographic photoreceptor according to claim 1, further comprising a curable compound.

10. The electrophotographic photoreceptor according to claim 9, wherein a mass ratio of the curable compound to the electron-transporting compound in the protective layer is 1.0 or less.

11. An electrophotographic photoreceptor cartridge comprising the electrophotographic photoreceptor according to claim 1.

12. An image formation device comprising the electrophotographic photoreceptor according to claim 1.

13. A coating liquid for forming an electrophotographic photoreceptor protective layer comprising an electron-transporting compound represented by the following formula (1) and a solvent, 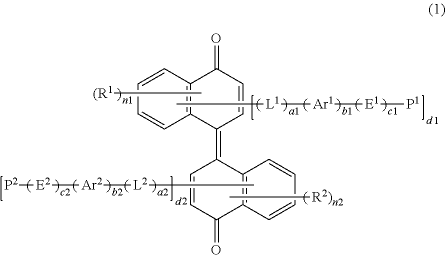
in formula (1),
L¹ and L² each independently represent a divalent group,
Ar¹ and Ar² each independently represent a divalent aromatic group having 6 to 30 carbon atoms or a divalent heteroaromatic group having 3 to 30 carbon atoms,
E¹ and E² each independently represent a divalent group,
P¹ and P² each independently represent a chain polymerizable functional group,
R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents,
a1, a2, b1, b2, c1 and c2 each independently represent an integer of 1 or more,
d1 and d2 each independently represent an integer of 0 or more and 6 or less, wherein d1+d2 is one or more, and n1 and n2 each independently represent an integer of 0 or more.

14. The coating liquid for forming an electrophotographic photoreceptor protective layer according to claim 13 further comprising a curable compound, wherein the curable compound content is 10 parts by mass or less based on 100 parts by mass of the solvent.

15. A compound represented by the following formula (1), 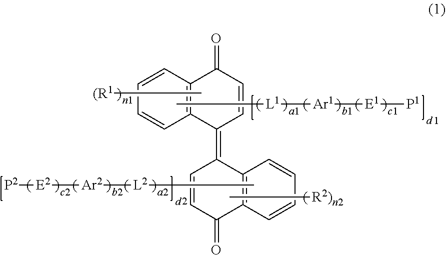
in formula (1),
L¹ and L² each independently represent a divalent group, Ar¹ and Ar² each independently represent a divalent aromatic group having 6 to 30 carbon atoms or a divalent heteroaromatic group having 3 to 30 carbon atoms,
E¹ and E² each independently represent a divalent group,
P¹ and P² each independently represent a chain polymerizable functional group,
R¹ and R² each independently represent an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, a halogen atom, a cyano group, an aromatic hydrocarbon group which may have one or more substituents or an aromatic heterocyclic group which may have one or more substituents,
a1, a2, b1, b2, c1 and c2 each independently represent an integer of 1 or more,
d1 and d2 each independently represent an integer of 0 or more and 6 or less, wherein d1+d2 is one or more, and n1 and n2 each independently represent an integer of 0 or more.

16. The compound according to claim 15, wherein P¹ and P² in formula (1) are each independently an acryloyl group or a methacryloyl group.

17. The compound according to claim 15, wherein Ar¹ and Ar² in formula (1) are each independently a phenylene group, a naphthylene group or a pyridylene group.

18. The compound according to claim 15, wherein L′ and L² in formula (1) are each independently an alkylene group.

19. The compound according to claim 15, wherein E¹ and E² in formula (1) are each independently a divalent group having an ester bond.

20. The compound according to claim 19, wherein E¹ and E² in formula (1) are each independently a divalent group having two or more ester bonds.

21. The compound according to claim 20, wherein the divalent group having two or more ester bonds comprises a structure represented by formula (E-1) or the formula (E-2) [chemical structure]
